(19) United States
(12) Patent Application Publication
(10) Pub. No.: US 2026/0192085 A1
(43) Pub. Date: Jul. 9, 2026

(54) Intermittent-Catheter Systems and Assemblies as Well as Methods Thereof

(71) Applicant: C. R. Bard, Inc., Franklin Lakes, NJ (US)

(72) Inventors: Pooja Bhuvanesh Kulkarni, Pune (IN); Sudev Geetha Surendran, Kozhikode District (IN); James David Hughett, Sr., Monroe, GA (US); Ronald N. Legaspi, Alpharetta, GA (US)

(21) Appl. No.: 19/131,882

(22) PCT Filed: Nov. 21, 2023
(86) PCT No.: PCT/US2023/080769

§ 371 Date: May 21, 2025

Related U.S. Application Data
(60) Provisional application No. 63/427,030, filed on Nov. 21, 2022.

(51) Int. Cl. A61M 25/00

(52) U.S. Cl. CPC A61M 25/002; A61M 25/0045; A61M 25/0068; A61M 2025/0046; A61M 2202/0014; A61M 2202/0496; A61M 2205/0222; A61M 2205/0238; A61M 2205/586; A61M 2209/06; A61M 2210/1092

(57) ABSTRACT

An intermittent-catheter system includes a rigid catheter housing (750) and an intermittent catheter (230). The catheter housing can include an inner sleeve (752), an outer sleeve (754) slidably mounted over the inner sleeve, and an end cap (756) configured to sit within an end portion of the outer sleeve and contribute to both a primary chamber (758) and a secondary chamber (760). The intermittent-catheter system can have a storage state in which the catheter housing includes a maximally extended state thereof with the intermittent catheter sealed within the primary chamber. The intermittent-catheter system can have an opened state in which the catheter housing includes a maximally collapsed state thereof with the end cap popped out of the outer sleeve exposing a funnel (236) of the intermittent catheter for removal of the intermittent catheter from the catheter housing.

### PRIORITY

This application claims the benefit of priority to U.S. Provisional Application No. 63/427,030, filed Nov. 21, 2022, which is incorporated by reference in its entirety into this application.

### CROSS-REFERENCE

This application is related to PCT/US2021/043771, filed Jul. 29, 2021; PCT/US2021/044021, filed Jul. 30, 2021; and PCT/US2021/049867, filed Sep. 10, 2021, each of which is incorporated by reference in its entirety into this application.

### BACKGROUND

Users of urinary catheters such as intermittent catheters self-catheterize four to six times a day. However, catheterization with such intermittent catheters can be messy. Indeed, existing intermittent catheters often include water or lubricant in packages thereof for lubrication of the intermittent catheters prior to insertion, but such packages are not configured to keep the water or lubricant from making messes. That, and such packages are not configured to keep post-catheterization messes from occurring such as those involving voided urine. What is needed are simple-to-use intermittent catheters that facilitate cleanliness before and after use.

Disclosed herein are intermittent-catheter systems and assemblies as well as methods thereof that address the foregoing.

### SUMMARY

Also disclosed herein is an intermittent-catheter system including, in some embodiments, a rigid catheter housing and an intermittent catheter. The catheter housing includes an inner sleeve, an outer sleeve slidably mounted over the inner sleeve, and an end cap. The end cap is configured to sit within an end portion of the outer sleeve opposite a closed end portion of the inner sleeve and simultaneously contribute to both a primary chamber for primary containment and a secondary chamber for secondary containment. The intermittent catheter includes a catheter tube and a funnel over a proximal end portion of the catheter tube. The intermittent-catheter system has a storage state and an opened state. In the storage state, the catheter housing can be at a maximally extended state thereof with the intermittent catheter sealed within the primary chamber. In the opened state, the outer sleeve of the catheter housing is slid over the inner sleeve of the catheter housing such that the catheter housing can be at a maximally collapsed state thereof with the end cap popped out of the outer sleeve and the funnel of the intermittent catheter exposed for removal of the intermittent catheter from the catheter housing.

In some embodiments, the end cap overlaps an open-end portion of the inner sleeve, thereby providing the primary chamber between an end-cap cavity of the end cap and an inner-sleeve cavity of the inner sleeve.

In some embodiments, an end-cap gasket around the end cap and an inner-sleeve gasket around the inner sleeve define longitudinal limits of the secondary chamber between an interior surface of the outer sleeve and exterior surfaces of the end cap and the inner sleeve.

In some embodiments, the primary chamber and the secondary chamber respectively provide the primary containment and the secondary containment for any residual urine in or around the intermittent catheter, thereby preventing urine leakage from the catheter housing in the storage state of the intermittent-catheter system after use of the intermittent catheter.

In some embodiments, the end-cap gasket and the inner-sleeve gasket provide sufficient friction for maintaining the storage state of the catheter housing, thereby averting inadvertent collapse into the opened state of catheter housing.

In some embodiments, an inner perimeter of an outer-sleeve cavity of the outer sleeve steps down in magnitude with an outer-sleeve step toward an opposite end portion of the outer sleeve to that including the end cap. The inner sleeve includes an external ridge configured to abut the outer-sleeve step in the opposite end portion of the outer sleeve, thereby defining the maximally extended state of the catheter housing.

In some embodiments, an outer perimeter of the inner sleeve steps up in magnitude with an inner-sleeve step toward the closed end portion of the inner sleeve. A taper of the inner perimeter of the outer-sleeve cavity of the outer sleeve is configured to restrict movement of the inner sleeve through the end portion of the outer sleeve including the end cap, thereby defining the maximally collapsed state of the catheter housing.

In some embodiments, the inner-sleeve step in the outer perimeter of the inner sleeve centers the inner sleeve in the outer sleeve when transitioning between the storage state and the opened state of the catheter housing.

In some embodiments, the intermittent-catheter system further includes a lubricious coating on or a lubricant around the catheter tube of the intermittent catheter sealed within the primary chamber in the storage state of the intermittent-catheter system.

In some embodiments, the primary chamber and the secondary chamber respectively provide the primary containment and the secondary containment for the lubricant around the catheter tube of the intermittent catheter, thereby preventing lubricant leakage from the catheter housing in the storage state of the intermittent-catheter system before use of the intermittent catheter.

In some embodiments, the catheter housing is configured to collapse when the closed end portion of the inner sleeve is held against a surface and the outer sleeve is slid over the inner sleeve toward the surface, thereby exposing the funnel of the intermittent catheter for removal of the intermittent catheter from the catheter housing.

In some embodiments, the funnel includes a plurality of ridges integrated into an outer surface of the funnel. The ridges are configured for gripping the funnel as a handle while removing the intermittent catheter from the catheter housing or voiding urine.

In some embodiments, the catheter tube includes a plurality of eyelets proximate a catheter tip. The eyelets are in fluid communication with the funnel opening.

In some embodiments, the intermittent catheter is configured for use by female humans.

Also disclosed herein is a method of an intermittent-catheter system. The method includes, in some embodiments, obtaining the intermittent-catheter system in a storage state thereof. The intermittent-catheter system includes a rigid catheter housing and an intermittent catheter. The catheter housing includes an inner sleeve, an outer sleeve slidably mounted over the inner sleeve, and an end cap. The end cap is configured to sit within an end portion of the outer sleeve opposite a closed end portion of the inner sleeve and simultaneously contribute to both a primary chamber for primary containment and a secondary chamber for secondary containment. The intermittent catheter includes a catheter tube and a funnel over a proximal end portion of the catheter tube. The catheter housing can be at a maximally extended state thereof with the intermittent catheter sealed within the primary chamber in the storage state of the intermittent-catheter system. The method also includes transitioning the intermittent-catheter system into an opened state thereof. Transitioning the intermittent-catheter system into the opened state includes grasping an outer sleeve of the catheter housing; holding a closed end portion of an inner sleeve of the catheter housing against a surface by way of the outer sleeve of the catheter housing; and sliding the outer sleeve of the catheter housing over the inner sleeve of the catheter housing toward the surface to expose a funnel of the intermittent catheter. The method also includes removing the intermittent catheter from the catheter housing by the funnel of the intermittent catheter.

In some embodiments, the funnel of the intermittent catheter pops the end cap out of the outer sleeve of the catheter housing when sliding the outer sleeve over the inner sleeve of the catheter housing.

In some embodiments, the end cap overlaps an open-end portion of the inner sleeve, thereby providing the primary chamber between an end-cap cavity of the end cap and an inner-sleeve cavity of the inner sleeve.

In some embodiments, an end-cap gasket around the end cap and an inner-sleeve gasket around the inner sleeve define longitudinal limits of the secondary chamber between an interior surface of the outer sleeve and exterior surfaces of the end cap and the inner sleeve.

In some embodiments, the primary chamber and the secondary chamber respectively provide the primary containment and the secondary containment for any lubricant around the catheter tube of the intermittent catheter, thereby preventing lubricant leakage from the catheter housing in the storage state of the intermittent-catheter system before use of the intermittent catheter.

In some embodiments, the method further includes inserting a distal end portion of the catheter tube into a urethra; voiding urine from a bladder through the intermittent catheter; replacing the intermittent catheter in the inner sleeve of the catheter housing; and replacing the end cap within the end portion of the outer sleeve, thereby reforming the primary chamber for primary containment of any residual urine in or around the intermittent catheter and the secondary chamber for secondary containment of any leaked urine from the primary chamber. Reforming the primary chamber and the secondary chamber thusly prevents urine leakage from the catheter housing in the storage state of the intermittent-catheter system after use of the intermittent catheter.

These and other features of the concepts provided herein will become more apparent to those of skill in the art in view of the accompanying drawings and following description, which describe particular embodiments of such concepts in greater detail.

### DRAWINGS

FIG. 1 illustrates an exploded view of a first intermittent-catheter system including a drainage assembly and a first rigid carrying case in an unused state of the first intermittent-catheter system in accordance with some embodiments.

FIG. 2 illustrates an exploded view of the drainage assembly of the first intermittent-catheter system of FIG. 1 in accordance with some embodiments.

FIG. 3 illustrates an exploded view of the first intermittent-catheter system in a used state of the first intermittent-catheter system in accordance with some embodiments.

FIG. 4 illustrates an exploded view of a first intermittent-catheter assembly of the drainage assembly including a first intermittent catheter and a storage sheath in accordance with some embodiments.

FIG. 5 illustrates a side view of the first intermittent catheter in accordance with some embodiments.

FIG. 6 illustrates a longitudinal cross section of the first intermittent catheter in accordance with some embodiments.

FIG. 7 illustrates a detailed view of the longitudinal cross section of the first intermittent catheter of FIG. 6 in accordance with some embodiments.

FIG. 8 illustrates a side view of the first intermittent-catheter assembly in accordance with some embodiments.

FIG. 9 illustrates a longitudinal cross section of the first intermittent-catheter assembly in accordance with some embodiments.

FIG. 10 illustrates a detailed view of the longitudinal cross section of the first intermittent-catheter assembly of FIG. 9 in accordance with some embodiments.

FIG. 11 illustrates another detailed view of the longitudinal cross section of the first intermittent-catheter assembly of FIG. 9 in accordance with some embodiments.

FIG. 12 illustrates an isometric view of a second intermittent-catheter system in a storage state in accordance with some embodiments.

FIG. 13 illustrates an exploded view of the second intermittent-catheter system in accordance with some embodiments.

FIG. 14 illustrates a side view of the second intermittent-catheter system in accordance with some embodiments.

FIG. 15 illustrates a longitudinal cross section of the second intermittent-catheter system in accordance with some embodiments.

FIG. 16 illustrates a side view of a second intermittent catheter of the second intermittent-catheter system in accordance with some embodiments.

FIG. 17 illustrates a longitudinal cross section of the second intermittent catheter in accordance with some embodiments.

FIG. 18 illustrates a detailed view of the longitudinal cross section of the second intermittent catheter of FIG. 17 in accordance with some embodiments.

FIG. 19 illustrates a side view of an outer sleeve of a first rigid catheter housing of the second intermittent-catheter system in accordance with some embodiments.

FIG. 20 illustrates a longitudinal cross section of the outer sleeve of the first catheter housing in accordance with some embodiments.

FIG. 21 illustrates a transverse cross section of the outer sleeve of the first catheter housing in accordance with some embodiments.

FIG. 22 illustrates another transverse cross section of the outer sleeve of the first catheter housing in accordance with some embodiments.

FIG. 23 illustrates a second intermittent-catheter assembly in an unused state in accordance with some embodiments.

FIG. 24 illustrates an opening mechanism for opening the second intermittent-catheter assembly in accordance with some embodiments.

FIG. 25A illustrates a sealing mechanism for sealing the second intermittent-catheter assembly after use in accordance with some embodiments.

FIG. 25B illustrates a funnel of a third intermittent catheter of the second intermittent-catheter assembly including a rigid insert in accordance with some embodiments.

FIG. 26 illustrates a drainage bag having an integrated fold-sealed disposal pocket in an unfolded state of the drainage bag in accordance with some embodiments.

FIG. 27 illustrates an unfolding mechanism for unfolding the drainage bag from a folded state in accordance with some embodiments.

FIG. 28 illustrates a first tear tab for creating a urine outlet for draining any urine from the drainage bag in accordance with some embodiments.

FIG. 29 illustrates a second tear tab for creating the urine outlet for draining any urine from the drainage bag in accordance with some embodiments.

FIG. 30 illustrates a third tear tab for creating the urine outlet for draining any urine from the drainage bag in accordance with some embodiments.

FIG. 31 illustrates a pop-up stoppered spout for the urine outlet for draining any urine from the drainage bag in accordance with some embodiments.

FIG. 32 illustrates a rip cord for creating the urine outlet for draining any urine from the drainage bag in accordance with some embodiments.

FIG. 33 illustrates a screw cap-covered spout for the urine outlet for draining any urine from the drainage bag in accordance with some embodiments.

FIG. 34 illustrates a hinged lid-covered spout for the urine outlet for draining any urine from the drainage bag in accordance with some embodiments.

FIG. 35 illustrates a first pair of peal-and-seal tabs for creating the urine outlet for draining any urine from the drainage bag in accordance with some embodiments.

FIG. 36 illustrates a second pair of peal-and-seal tabs for creating the urine outlet for draining any urine from the drainage bag in accordance with some embodiments.

FIG. 37 illustrates a membrane-covered spout for the urine outlet for draining any urine from the drainage bag in accordance with some embodiments.

FIG. 38 illustrates a piercing element for piercing the membrane-covered spout for draining any urine from the drainage bag in accordance with some embodiments.

FIG. 39 illustrates a peal-and-seal sticker for opening the urine outlet for draining any urine from the drainage bag in accordance with some embodiments.

FIG. 40 illustrates the drainage bag having a detachable fold-sealed disposal pouch in an unfolded state of the drainage bag in accordance with some embodiments.

FIG. 41 illustrates the drainage bag having a detachable zipper-sealed disposal pouch in an unfolded state of the drainage bag in accordance with some embodiments.

FIG. 42 illustrates a second rigid carrying case of the first intermittent-catheter system in a closed state of the second carrying case in accordance with some embodiments.

FIG. 43 illustrates the second carrying case in an opened state in accordance with some embodiments.

FIG. 44 illustrates an anterior face of a third rigid carrying case of the first intermittent-catheter system in a closed state of the third carrying case in accordance with some embodiments.

FIG. 45 illustrates a posterior face of the third carrying case in the closed state in accordance with some embodiments.

FIG. 46 illustrates the third carrying case in an opened state in accordance with some embodiments.

FIG. 47 illustrates an isometric view of a third intermittent-catheter system including a third intermittent-catheter assembly in a second catheter housing as well as an opening mechanism for opening the second catheter housing in accordance with some embodiments.

FIG. 48 illustrates a side view of the opening mechanism for opening the second catheter housing and removing the third intermittent-catheter assembly from the second catheter housing in accordance with some embodiments.

FIG. 49 illustrates an isometric view of third intermittent-catheter system including the third intermittent-catheter assembly in a third catheter housing as well as an opening mechanism for opening the third catheter housing in accordance with some embodiments.

FIG. 50 illustrates a side view of the opening mechanism for opening the third catheter housing and removing the third intermittent-catheter assembly from the third catheter housing in accordance with some embodiments.

FIG. 51 illustrates an isometric view of a fourth intermittent-catheter system including the third intermittent-catheter assembly in a fourth rigid carrying case as well as an opening mechanism for opening the fourth carrying case in accordance with some embodiments.

FIG. 52 illustrates an isometric view of the fourth intermittent-catheter system including the third intermittent-catheter assembly in a fifth rigid carrying case as well as an opening mechanism for opening the fifth carrying case in accordance with some embodiments.

FIG. 53 illustrates an isometric view of a fifth intermittent-catheter system including a first tray package in an unused state of the fifth intermittent-catheter system in accordance with some embodiments.

FIG. 54 illustrates an isometric view of the fifth intermittent-catheter system including the drainage assembly in an opened state of the fifth intermittent-catheter system in accordance with some embodiments.

FIG. 55 illustrates a securing mechanism of the first tray package for securing the fifth intermittent-catheter system during a catheterization procedure in accordance with some embodiments.

FIG. 56 illustrates an isometric view of the fifth intermittent-catheter system including a second tray package in an unused state of the fifth intermittent-catheter system in accordance with some embodiments.

FIG. 57 illustrates a securing mechanism of the second tray package for securing the fifth intermittent-catheter system during a catheterization procedure in accordance with some embodiments.

FIG. 58 illustrates an anterior view of a third tray package of the fifth intermittent-catheter system in an unused state of the third tray package in accordance with some embodiments.

FIG. 59 illustrates a posterior view of the third tray package in the unused state in accordance with some embodiments.

FIG. 60 illustrates an opening-and-resealing mechanism for opening the third tray package and resealing the third tray package after a catheterization procedure in with some embodiments.

FIG. 61 illustrates an anterior view of a sixth rigid carrying case of the first intermittent-catheter system in an unused state of the sixth carrying case in accordance with some embodiments.

FIG. 62 illustrates an anterior view of the sixth carrying case in an opened state of the sixth carrying case in accordance with some embodiments.

FIG. 63 illustrates an anterior view of the sixth carrying case in a resealed state of the sixth carrying case in accordance with some embodiments.

FIG. 64 illustrates a posterior view of the sixth carrying case in the unused or resealed state of the sixth carrying case in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have components or features that can be readily separated from the particular embodiment and optionally combined with or substituted for components or features of any of a number of other embodiments disclosed herein. In an example, any intermittent-catheter system disclosed herein can be modified to include the carrying case, tray package, or catheter housing; the drainage assembly; the intermittent-catheter assembly; the intermittent catheter; or any other component thereof of another intermittent-catheter system disclosed herein unless context or reason suggests otherwise.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

An unused state of an intermittent-catheter system or any component thereof indicates the intermittent-catheter system or component thereof being described remains packaged as manufactured, distributed, or sold.

A used state of an intermittent-catheter system or any component thereof indicates the intermittent-catheter system or component thereof being described is not in an unused state; that is, the intermittent-catheter system or the component thereof is opened, unpackaged, prepared for use in a catheterization procedure, used in such a catheterization procedure, or the like.

A storage state of an intermittent-catheter system or any component thereof indicates the intermittent-catheter system or component thereof being described has been opened, unpackaged, prepared for use in a catheterization procedure, used in such a catheterization procedure, or the like but repackaged in some manner for storage until disposal is possible. Alternatively, the storage state of an intermittent-catheter system or any component thereof can indicate the intermittent-catheter system or component thereof being described remains packaged as manufactured, distributed, or sold per the unused state. Therefore, context should be used to inform any interpretation of the storage state of the intermittent-catheter system or component thereof.

A closed state of an intermittent-catheter system or any component thereof indicates the intermittent-catheter system or component thereof being described is simply closed. As such, the intermittent-catheter system or component thereof being described can be in an unused state, a used state (but closed), or a storage state. Therefore, context should be used to inform any interpretation of the closed state of the intermittent-catheter system or component thereof beyond simply being closed.

An opened state of an intermittent-catheter system or any component thereof indicates the intermittent-catheter system or component thereof being described is simply opened. As such, the intermittent-catheter system or component thereof being described is also in a used state, as above. Indeed, whether or not the intermittent-catheter system or any component thereof has been prepared for use in a catheterization procedure or used in such a catheterization procedure, sterility of the intermittent-catheter system is potentially compromised.

A carrying case or tray package of an intermittent-catheter system is generally configured to enclose therein one or more drainage bags, one or more intermittent-catheter assemblies or intermittent catheters, and any other components for intermittent catheterization.

A catheter housing of an intermittent-catheter system is generally configured to enclose therein one or more intermittent-catheter assemblies or intermittent catheters. The catheter housing and any other components for intermittent catheterization including drainage bags can be enclosed in a carrying case or tray package disclosed herein or something of a user's choosing.

Unless defined otherwise, all technical and scientific terms used herein have the same meaning as commonly understood by those of ordinary skill in the art.

### Male or Unisex Intermittent-Catheter Systems

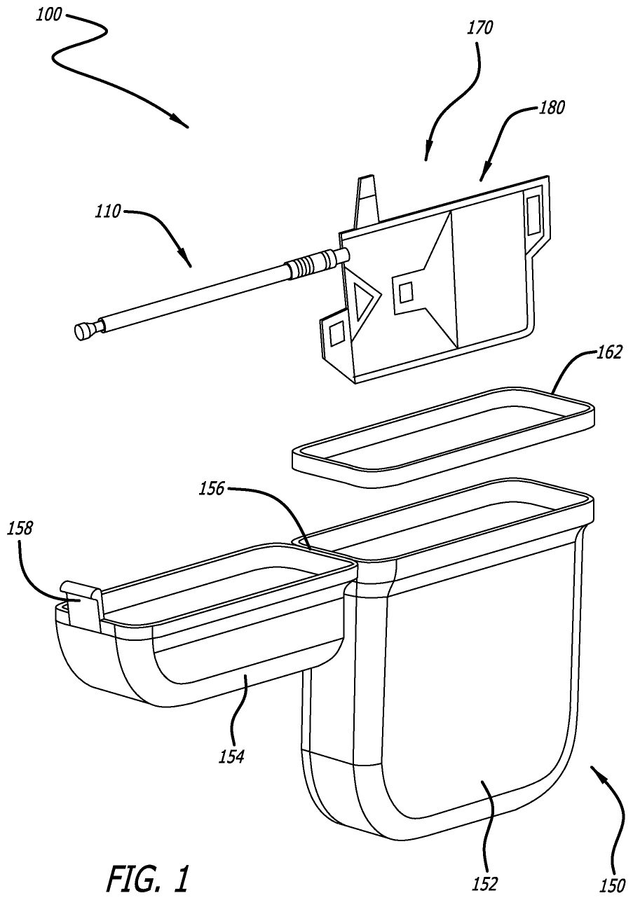

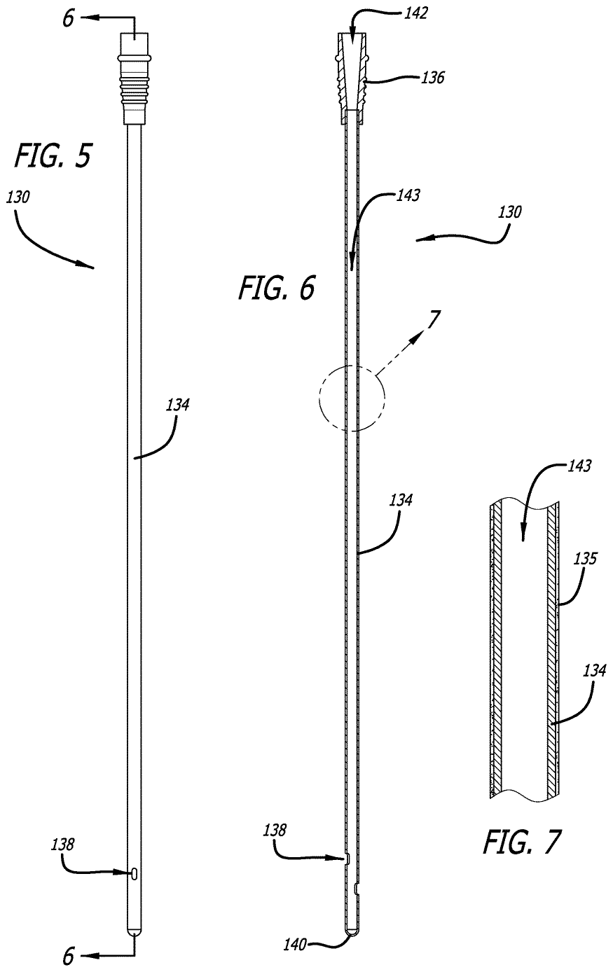

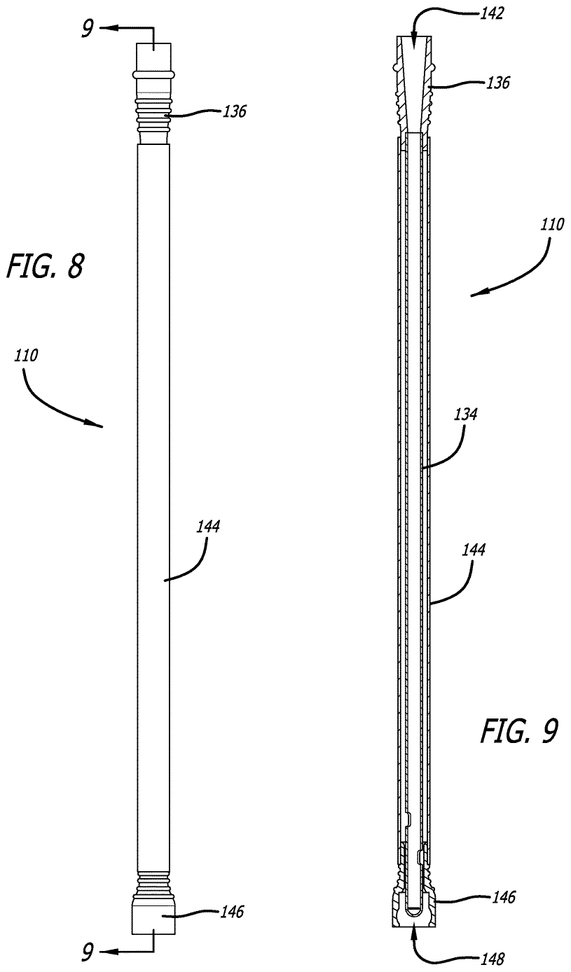

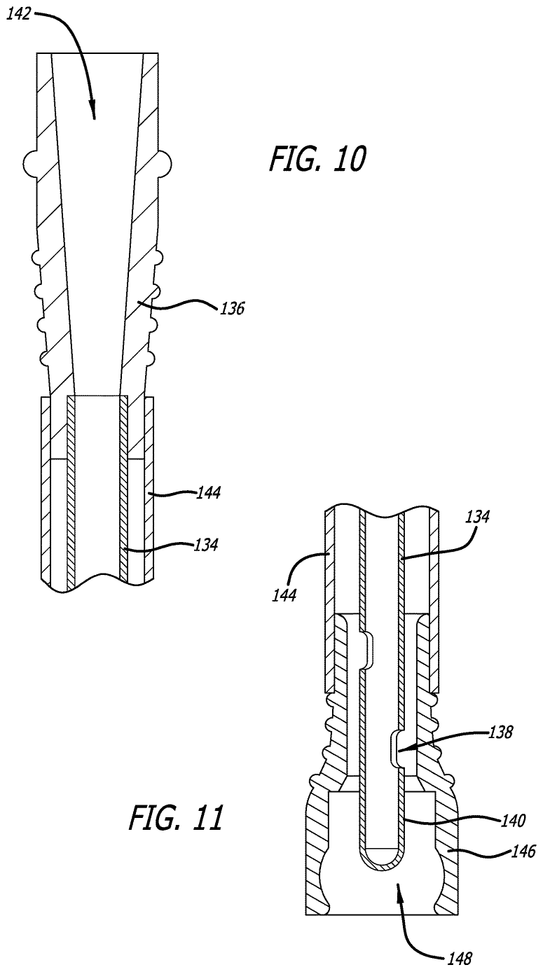

FIGS. 1-11, 23, 24, 25A, 25B, 26-64 illustrate various intermittent-catheter systems or components thereof in accordance with some embodiments. Such intermittent-catheter systems and components are configured for use by male humans or humans with non-specific anatomy.

Notably, the intermittent-catheter system 100 is set forth immediately below as a reference embodiment for describing the various intermittent-catheter systems and components thereof. It should be understood that other intermittent-catheter systems or their components set forth below might not be described in as much detail as the reference embodiment for such detail can be gleaned from description of the reference embodiment. Indeed, to reduce expository burden, description of the other intermittent-catheter systems or their components often focuses on differences from the reference embodiment of the intermittent-catheter system 100 and the components thereof instead of reiterating commonalities. Lastly, as set forth above, it should be understood that any intermittent-catheter system disclosed herein can be modified to include the carrying case, tray package, or catheter housing; the drainage assembly; the intermittent-catheter assembly; the intermittent catheter; or any other component thereof of another intermittent-catheter system disclosed herein unless context or reason suggests otherwise.

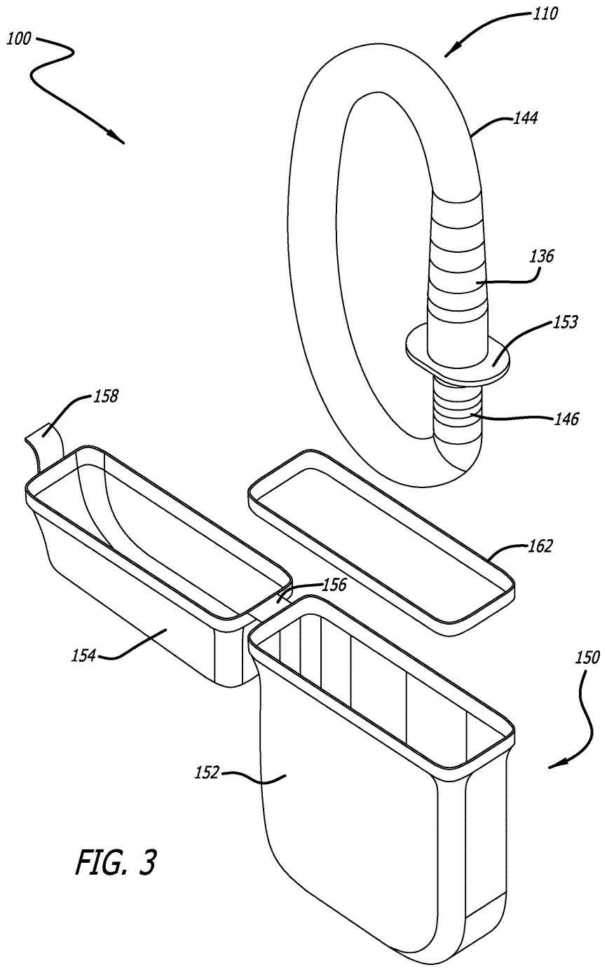

FIGS. 1-11 illustrate an intermittent-catheter system 100 in accordance with some embodiments. Notably, FIG. 1 illustrates the intermittent-catheter system 100 in an unused state of the intermittent-catheter system 100, and FIG. 3 illustrates the intermittent-catheter system 100 in a used state of the intermittent-catheter system 100.

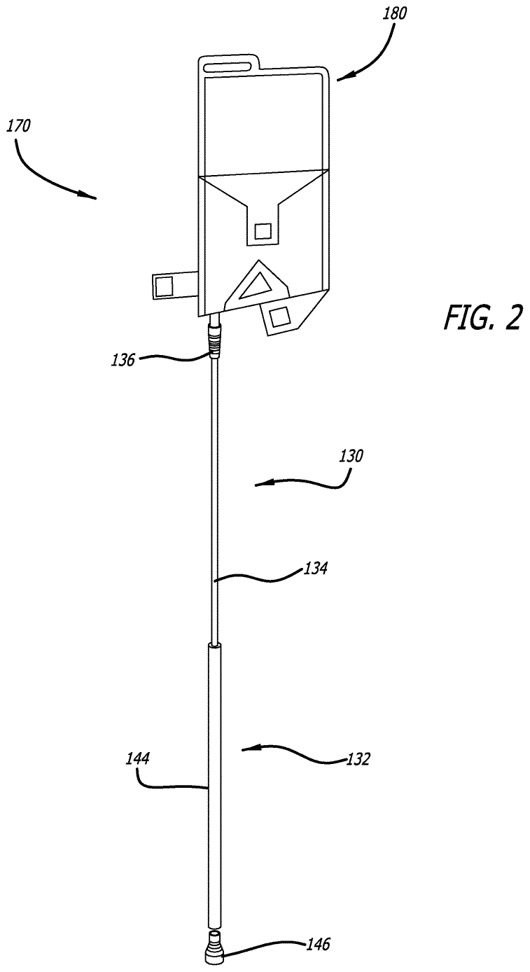

As shown, the intermittent-catheter system 100 can include a drainage assembly 170 and a rigid carrying case selected from the various carrying cases set forth below. The drainage assembly 170, in turn, can include an intermittent-catheter assembly 110 and a drainage bag 180, which drainage bag 180 can be pre-connected to the funnel 136 of the intermittent catheter 130 of the intermittent-catheter assembly 110 as shown in FIG. 2. If the drainage bag 180 is not pre-connected to the funnel 136 of the intermittent catheter 130 as shown in FIG. 2, the drainage assembly 170 can instead be referred to as drainage components of the intermittent-catheter system 100 or another intermittent-catheter system, as the case might be. Such drainage components include the intermittent-catheter assembly 110 and the intermittent catheter 130 thereof. Each component of the intermittent-catheter assembly 110, the drainage bag 180, and the carrying case 150 is set forth in more detail below.

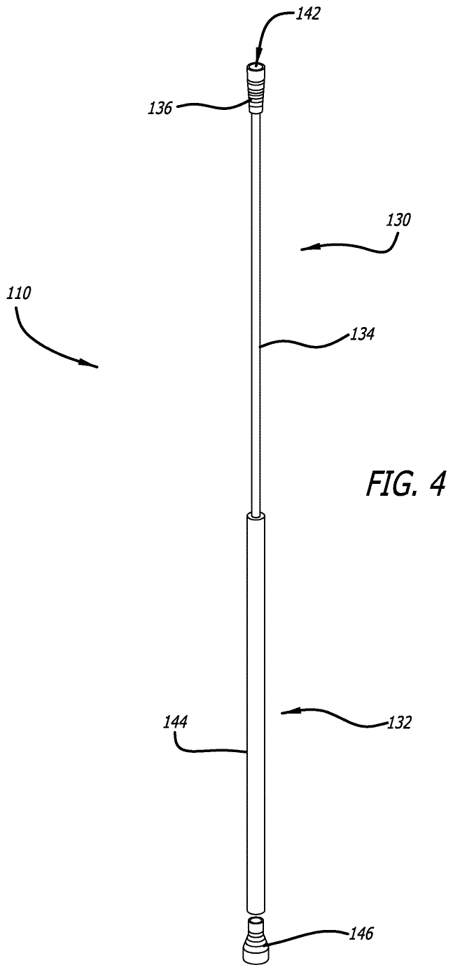

FIGS. 4-11 illustrate the intermittent-catheter assembly 110 in accordance with some embodiments.

As shown, the intermittent-catheter assembly 110 can include an intermittent catheter 130 and a sterility-preserving enclosure 132 therefor.

The intermittent catheter 130 can include a catheter tube 134 and a funnel 136 over a proximal end portion of the catheter tube 134. The catheter tube 134 can include a plurality of eyelets 138 proximate a catheter tip 140, and the funnel 136 can have a funnel opening 142 in fluid communication with the eyelets 138 by way of a lumen 143 through the intermittent catheter 130 for voiding urine.

The sterility-preserving enclosure 132 can include a storage sheath 144, an end cap 146 coupled to a distal end portion of the storage sheath 144, and, optionally, a tamper-proof, peelable seal over the cap 146 in at least the unused state of the intermittent-catheter assembly 110. The storage sheath 144 and end cap 146 are configured to enclose at least the catheter tube 134 of the intermittent catheter 130 therein to preserve its sterility in the unused state thereof. Indeed, the storage sheath 144 can extend between the funnel 136 of the intermittent catheter 130 and the end cap 146, and the end cap 146 can extend over a distal end of the catheter tube 134, thereby protecting the catheter tube 134 from contamination as well as keeping any lubricious coating 135 on the catheter tube 134 or any lubricant around the catheter tube 134 contained within the sterility-preserving enclosure 132 or storage sheath 144 thereof in the unused state of the intermittent-catheter assembly 110. However, it should be understood that the sterility-preserving enclosure 132 can also double as a urine-retaining enclosure, thereby keeping any residual urine in or around the catheter tube 134 contained within the urine-retaining enclosure or storage sheath 144 thereof in the used state of the intermittent-catheter assembly 110. Transitioning between the sterility-preserving enclosure 132 and the urine-retaining enclosure involves at least withdrawing the storage sheath 144 over the catheter tube 134, voiding urine through the intermittent catheter 130, and advancing the storage sheath 144 back over the catheter tube 134.

Notably, the intermittent-catheter assembly 110 can form a leak-free, ouroboros-type, end-to-end closure in the unused state of the intermittent-catheter assembly 110, the used state of the intermittent-catheter assembly 110, or both. Indeed, a distal portion of the end cap 146 can include a receptacle 148 configured to accept therein an end portion of the funnel 136 to form the end-to-end closure in the unused or used state of the intermittent-catheter assembly 110. Alternatively, the distal portion of the end cap 146 can include a plug (not shown) configured to insert into the funnel opening 142 of the funnel 136 to form the end-to-end closure in the unused or used state of the intermittent-catheter assembly 110. To assist in forming such an end-to-end closure, the end cap 146 can include a flange 153 configured to facilitate handling of the intermittent-catheter assembly 110 when forming the end-to-end closure in the used state of the intermittent-catheter assembly 110. Advantageously, the funnel 136 need not have a separate end cap to close the funnel 136 after use of the intermittent catheter 130. That, and formation of the end-to-end closure in the used state of the intermittent-catheter assembly 110 provides in a convenient, compact form of the used state of the intermittent-catheter assembly 110 until disposal is possible.

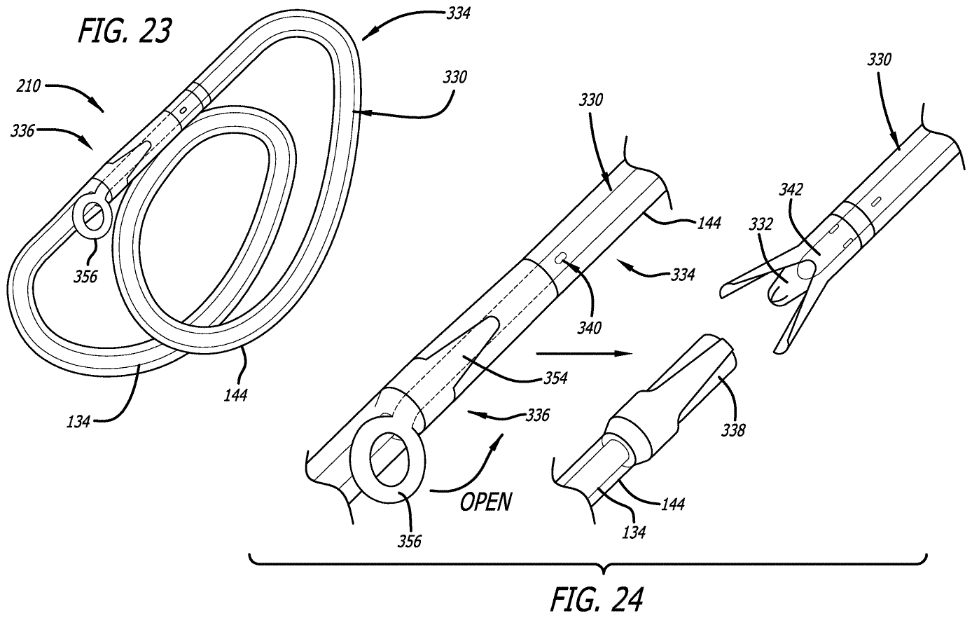

FIGS. 23, 24, 25A, and 25B illustrate an intermittent-catheter assembly 210 in accordance with some embodiments. Such an intermittent-catheter assembly 210 is an alternative to the intermittent-catheter assembly 110.

As shown, the intermittent-catheter assembly 210 can include an intermittent catheter 330, an introducer tip 332 over a distal end portion of the intermittent catheter 330, and a sterility-preserving enclosure 334 therefor. Notably, the sterility-preserving enclosure 334 of the intermittent-catheter assembly 210 precludes pre-connection of the intermittent-catheter assembly 210 and the drainage bag 180 such as in the unused state of the drainage assembly 170 set forth above because the introducer tip 332 and the funnel 338 of the intermittent catheter 330 form a leak-free, ouroboros-type, end-to-end closure in the unused state of the intermittent-catheter assembly 210, which is maintained by tamper-proof packaging 336.

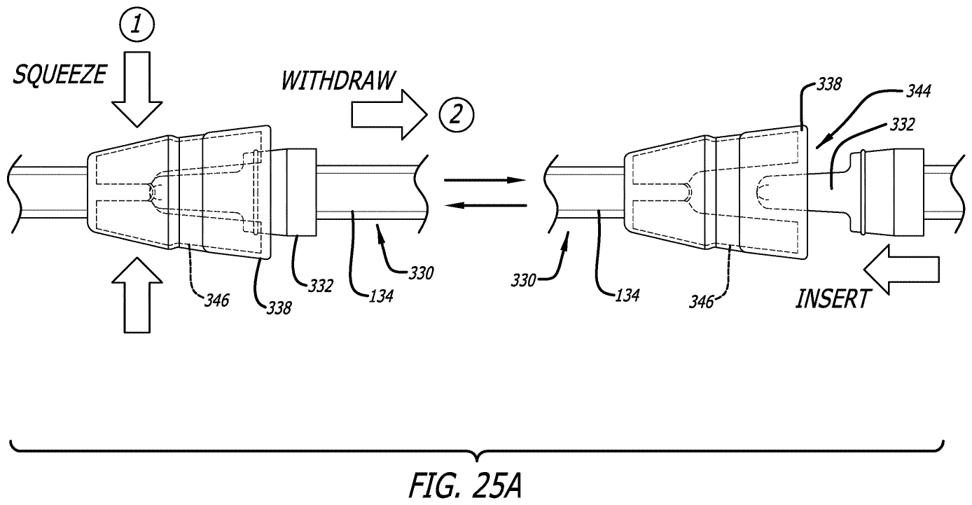

The intermittent catheter 330 can include a catheter tube 134 and a funnel 338 over a proximal end portion of the catheter tube 134. The catheter tube 134 can include a plurality of eyelets 340 proximate a catheter tip 342, and the funnel 338 can have a funnel opening 344 in fluid communication with the eyelets 340 by way of a lumen through the intermittent catheter 330 for voiding urine. Further, the funnel 338 can be formed of a polymeric material ranging from a soft polymeric material having a relatively low durometer up to a rigid polymeric material having a relatively high durometer; however, when the funnel 338 is formed of the soft polymeric material, the funnel 338 can include a rigid insert 346 inserted or otherwise disposed therein as shown in FIGS. 25A and 25B.

The rigid insert 346 is configured with two levered semiconical halves 348 to snap or clamp onto the introducer tip 332 in the end-to-end closure of the unused state of the intermittent-catheter assembly 210 as well as the used state of the intermittent-catheter assembly 210. The rigid insert 346 can include interlocking features with the introducer tip 332 such as one or more semiannular grooves, one or more semiannular ridges, or some combination thereof provided the interlocking features are complementary to those of the introducer tip 332. Advantageously, the rigid insert 346 can be configured such that when a distal end portion of the funnel 338 is pinched or squeezed distal of a pivot point 352 between the two semiconical halves 348 of the rigid insert 346, the two semiconical halves 348 of the rigid insert 346 separate allowing the introducer tip 332 to be separated or otherwise removed from the funnel 338 of the intermittent catheter 330.

The introducer tip 332 is configured for insertion into the urinary meatus, through which introducer tip 332 the catheter tube 134 of the intermittent catheter 330 is advanced. Such an introducer tip 332 has a greater diameter than the catheter tube 134 making catheterization with the introducer tip 332 easier for low-dexterity users. The introducer tip 332 can also be formed of a rigid polymeric material having a relatively high durometer compared to the catheter tube 134, which further makes catheterization with the introducer tip 332 easier for low-dexterity users. Notably, the rigid polymeric material of the introducer tip 332 also enables the two levered semiconical halves 348 of the rigid insert 346 to snap or clamp onto the introducer tip 332 in the end-to-end closure of the unused state of the intermittent-catheter assembly 210 as well as the used state of the intermittent-catheter assembly 210. Further, such an introducer tip 332 can include interlocking features with the rigid insert 346 such as one or more annular grooves, one or more annular ridges, or some combination thereof provided the interlocking features are complementary to those of the rigid insert 346, which interlocking feature further enable the two levered semiconical halves 348 of the rigid insert 346 to snap or clamp onto the introducer tip 332 in the end-to-end closure of the unused state of the intermittent-catheter assembly 210 as well as the used state of the intermittent-catheter assembly 210.

The sterility-preserving enclosure 334 can include the storage sheath 144 configured to enclose an entirety of the intermittent catheter 330 therein to preserve its sterility in the unused state thereof. Indeed, the storage sheath 144 can extend from a distal end portion of the funnel 338, about which a proximal end portion of the storage sheath 144 is coupled, over an entirety of the catheter tube 134, over the introducer tip 332 to which it can be coupled, over a proximal end portion of the funnel 338, and back to the distal end portion of the funnel 338. A distal end portion of the storage sheath 144 is coupled to the distal end portion of the funnel 338 or otherwise covers it by way of the tamper-proof packaging 336, thereby protecting the an entirety of the intermittent catheter 330 and the introducer tip 332 from contamination, as well as keeping any lubricious coating 135 on the catheter tube 134 or any lubricant around the catheter tube 134 contained within the sterility-preserving enclosure 334 or storage sheath 144 thereof in the unused state of the intermittent-catheter assembly 210.

The tamper-proof packaging 336 can include a rip cord or a tear strip 354 such as that formed of the polymeric material of the storage sheath 144, itself, between weakened (e.g., scored, perforated, etc.) separation lines of the storage sheath 144, wherein the rip cord or tear strip 354 is configured for pulling the rip cord or tear strip 354 across the storage sheath 144 to open the tamper-proof packaging 336. As shown, the rip cord or tear strip 354 can include a handle 356 coupled thereto having any convenient shape of a variety of shapes such as the ring-shaped handle 356 shown in FIG. 24; however, the rip cord or tear strip 354 need not include the handle 356 in some embodiments if provided with additional length for grasping or wrapping around one or more fingers.

The sterility-preserving enclosure 334 can also double as a urine-retaining enclosure, thereby keeping any residual urine in or around the catheter tube 134 contained within the urine-retaining enclosure or storage sheath 144 thereof in the used state of the intermittent-catheter assembly 210. Indeed, after using the intermittent catheter 330 for voiding urine, which bunches the storage sheath 144 about the distal end portion of the funnel 338 as the catheter tube 134 of the intermittent catheter 330 is advanced through the introducer tip 332, the storage sheath 144 can be replaced over the intermittent catheter 330 and the introducer tip 332 as in the unused state of the intermittent-catheter assembly 210. Further, the introducer tip 332 can be inserted into the funnel 338 including the rigid insert 346 such that the two levered semiconical halves 348 of the rigid insert 346 snap or clamp onto the introducer tip 332 in the end-to-end closure of the used state of the intermittent-catheter, thereby keeping any residual urine in or around the catheter tube 134 contained within the urine-retaining enclosure or storage sheath 144 thereof.

FIGS. 1, 2, and 26-41 illustrate various drainage bags or components thereof in accordance with some embodiments. Notably, while such drainage bags are set forth herein as components of the male or unisex intermittent-catheter systems, it should be understood that the female intermittent-catheter system set forth below can include any drainage bag of FIGS. 1, 2, and 26-41.

As shown, the drainage bag 180 can be a foldable drainage bag including a urine-drainage means for draining any urine from the drainage bag 180 and a disposal means for disposing the drainage bag 180.

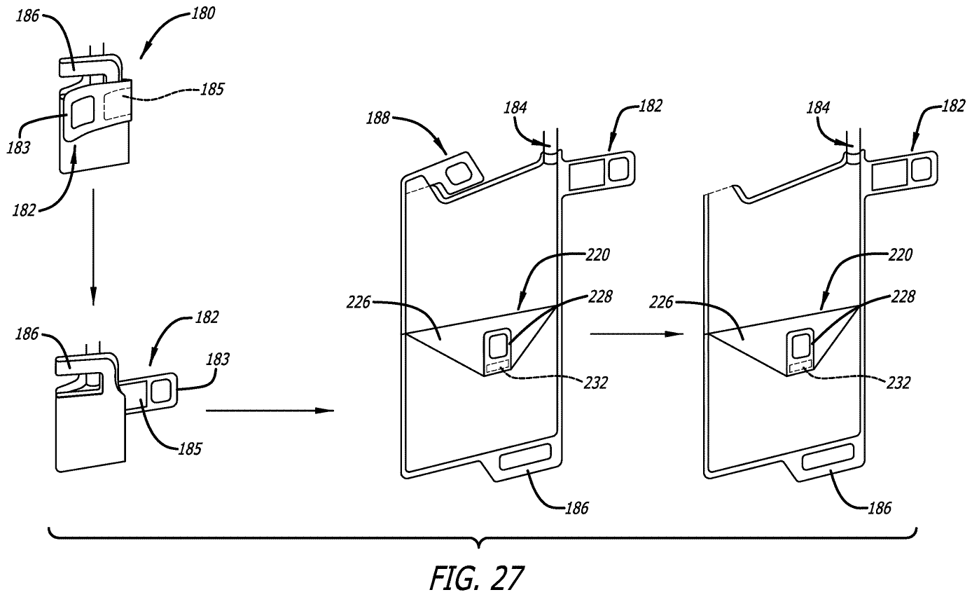

The drainage bag 180 can be in a folded state in the unused state of the drainage assembly 170 or the drainage components, which provides a compact form of the drainage bag 180 until it is used during a catheterization procedure. In the folded state of the drainage bag 180 shown in FIG. 27, the drainage bag 180 can include one or more folds along a width such that the drainage bag 180 has a shorter length in the folded state than in an unfolded state of the drainage bag 180, one or more folds along a length such that the drainage bag 180 has a shorter width in the folded state than in the unfolded state of the drainage bag 180, or some combination thereof. However, it should be understood that the drainage bag 180 can be folded into any convenient geometric shape (e.g., squares, rectangles, triangles, etc.) in the unused state of the drainage bag to suit it packaging in the rigid package set forth herein. The folded state of the drainage bag 180 can be maintained by a strap 182 extending from a longitudinal end portion of the drainage bag 180 also including a urine inlet 184 to which the intermittent catheter 130 of the intermittent-catheter assembly 110 connects. The strap 182 can include a handle 183 and an adhesive strip or patch 185 for adhering to a folded portion of the drainage bag 180 to maintain the folded state of the drainage bag 180. After peeling the strap 182 adhered to the folded portion of the drainage bag 180 away therefrom, the drainage bag 180 can be unfolded in accordance with an unfolding mechanism for unfolding the drainage bag 180 shown in FIG. 27. Notably, the drainage bag 180 can include a handle 186 extending from another longitudinal end portion of the drainage bag 180 opposite that including the urine inlet 184. The handle 186 is configured to facilitate unfolding the drainage bag 180 from the folded state of the drainage bag 180.

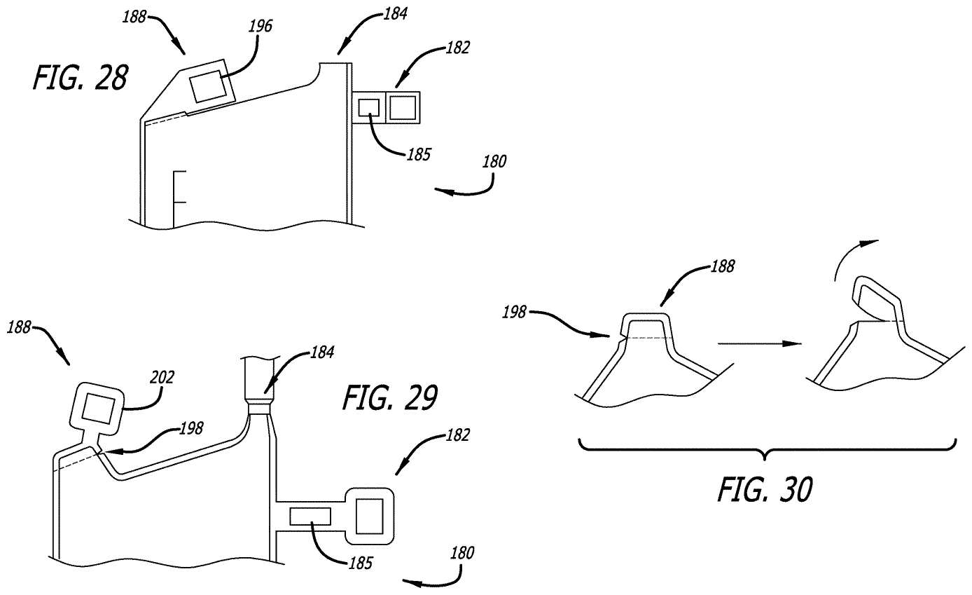

FIGS. 28-39 illustrate various urine-drainage means for draining any urine from the drainage bag 180 in accordance with some embodiments.

As shown, the urine-drainage means is opposite the strap 182 in the longitudinal end portion of the drainage bag 180 also including the urine inlet 184. Such a urine-drainage means can include a tear tab 188, a rip cord 190, peal-and-seal tabs 192, or a peal-and-seal sticker 194 for creating a urine outlet or a spout 195 selected from various spouts for the urine outlet.

FIGS. 28-30 provide examples of the tear tab 188 for creating the urine outlet, wherein the tear tab 188 of FIG. 28 includes a handle 196 configured for pulling the tear tab 188 directly across a weakened (e.g., scored, perforated, etc.) separation line of the drainage bag 180 starting from a starting notch 198 to create the urine outlet, the tear tab 188 of FIG. 29 includes a handle 202 configured for pulling the tear tab 188 out from the drainage bag 180 (e.g., away from the longitudinal end portion of the drainage bag 180 including the tear tab 188) and across the weakened separation line starting from the starting notch 198 to create the urine outlet, and the handleless tear tab 188 of FIG. 30 is configured for pulling the tear tab 188 directly across the weakened separation line of the drainage bag 180 starting from the starting notch 198 to create the urine outlet.

Similarly, FIG. 32 provides an example of the rip cord 190 for creating the urine outlet, wherein the rip cord 190 of FIG. 32 includes a ring-type handle 204 coupled thereto for pulling the rip cord 190 across a separation line of the drainage bag 180 under which the rip cord 190 is embedded to create the urine outlet. Notably, the handle 204 coupled to the rip cord 190 can be any convenient shape; however, the rip cord 190 need not include the handle 204 in some embodiments if provided with additional length for grasping or wrapping around one or more fingers.

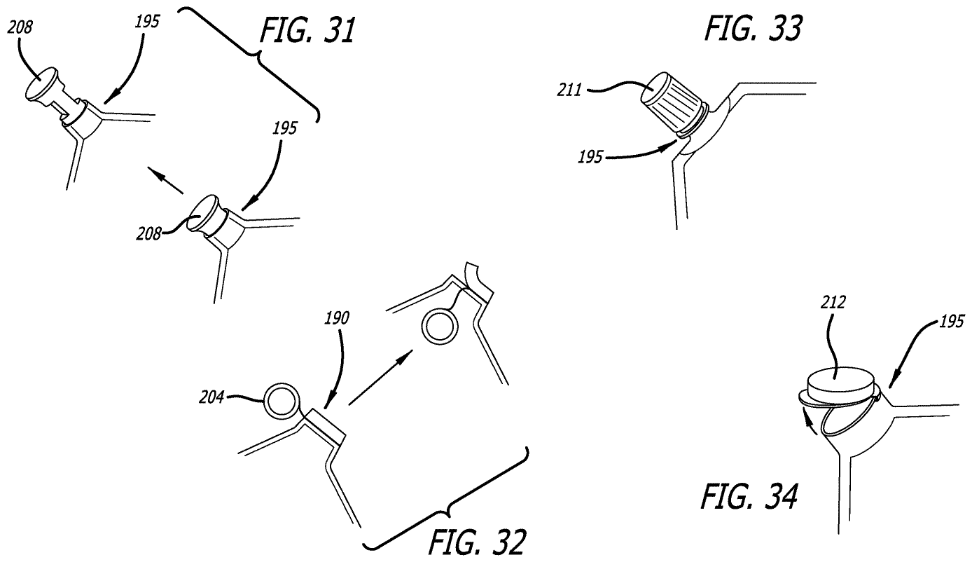

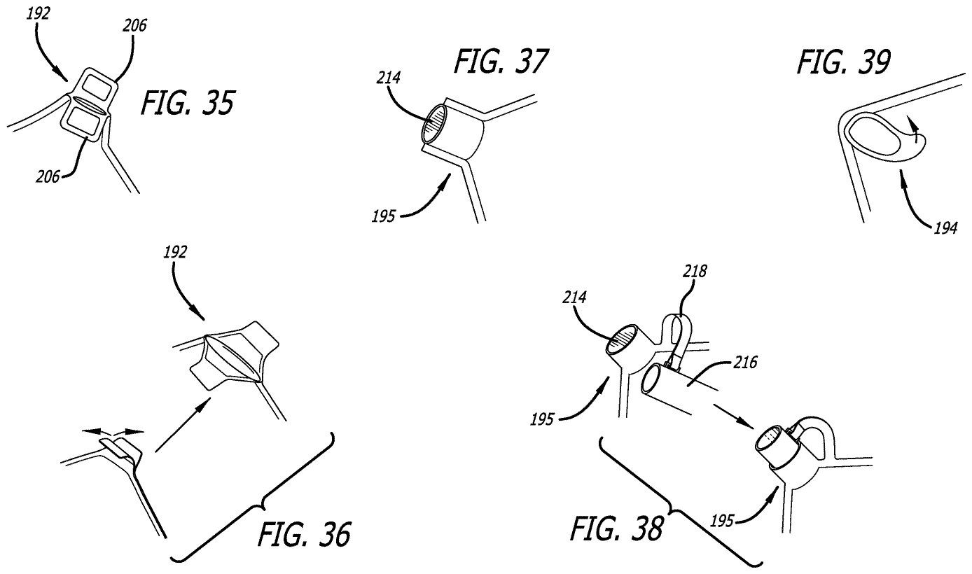

FIGS. 35 and 36 provide examples of the peal-and-seal tabs 192 for creating the urine outlet, wherein the peal-and-seal tabs 192 of FIG. 35 include handles 206 configured for pulling the peal-and-seal tabs 192 away from the drainage bag 180 (e.g., away from the anterior and posterior faces of the drainage bag 180) starting from a small separation between the peal-and-seal tabs 192 to create the urine outlet, and the handleless peal-and-seal tabs 192 of FIG. 36 are configured for pulling the peal-and-seal tabs 192 away from the drainage bag 180 (e.g., away from the anterior and posterior faces of the drainage bag 180) starting from a small separation between the peal-and-seal tabs 192 to create the urine outlet. Notably, the peal-and-seal tabs 192 are configured to reseal the urine outlet when the peal-and-seal tabs 192 are pressed back together; however, in some embodiments, the peal-and-seal tabs 192 can simply be peal tabs without an ability to reseal when pressed together.

Similarly, FIG. 39 provides an example of the peal-and-seal sticker 194 for creating the urine outlet, wherein the handleless peal-and-seal sticker 194 of FIG. 39 is configured for pulling the peal-and-seal sticker 194 away from the drainage bag 180 (e.g., away from the anterior or posterior face of the drainage bag 180) to create the urine outlet. Notably, the peal-and-seal sticker 194 is configured to reseal the urine outlet when the peal-and-seal sticker 194 is pressed back onto the drainage bag 180 over the urine outlet; however, in some embodiments, the peal-and-seal sticker 194 can simply be a peal sticker without an ability to reseal when pressed onto the drainage bag 180 over the urine outlet.

FIGS. 31, 33, 34, and 37 provide examples of the various spouts for the urine outlet, wherein the spout 195 of FIG. 31 includes a pop-up stopper 208 for a resealable pop-up stoppered spout, the spout 195 of FIG. 33 includes a screw cap 211 for a resealable screw cap-covered spout, the spout 195 of FIG. 34 includes a hinged lid 212 for a resealable hinged lid-covered spout, and the spout 195 of FIG. 37 includes a membrane 214 (e.g., a plastic membrane, a foil membrane, a composite membrane of the foregoing, etc.) for a membrane-covered spout. Notably, FIG. 38 provides an example of a piercing element 216 having a piercing end portion for piercing the membrane 214 of the membrane-covered spout for draining any urine from the drainage bag 180. As shown, such a piercing element 216 can be tethered to the drainage bag 180 with a tether 218 for retaining the piercing element 216 with the drainage bag 180. Further, as shown, the piercing element 216 can be an open-ended tube having a length sufficient to extend into the spout 195 beyond any broken pieces of the membrane 214 attached to a lip of the spout 195, thereby providing a smooth surface through the urine outlet for draining any urine therefrom. Alternatively, the piercing element 216 can be a closed-ended tube having a closed end opposite the piercing end portion of the piercing element 216, thereby providing a stopper for resealing the membrane-covered spout.

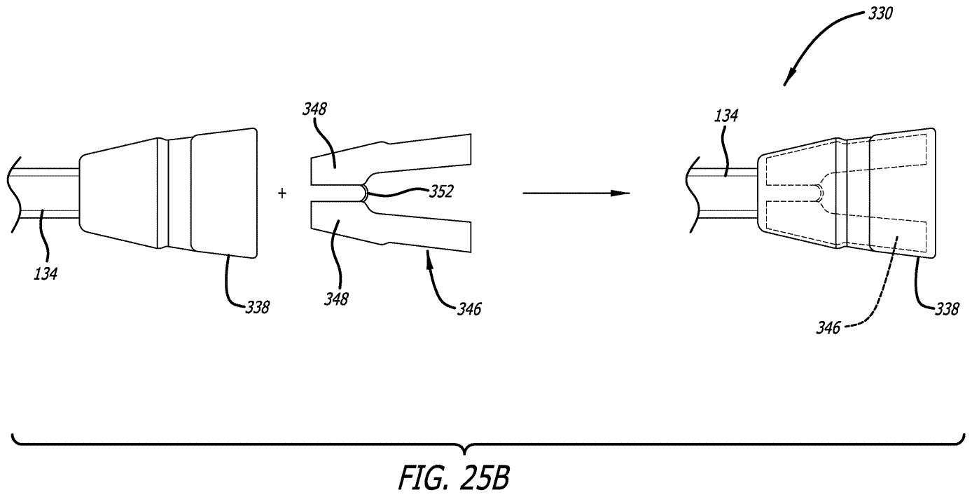

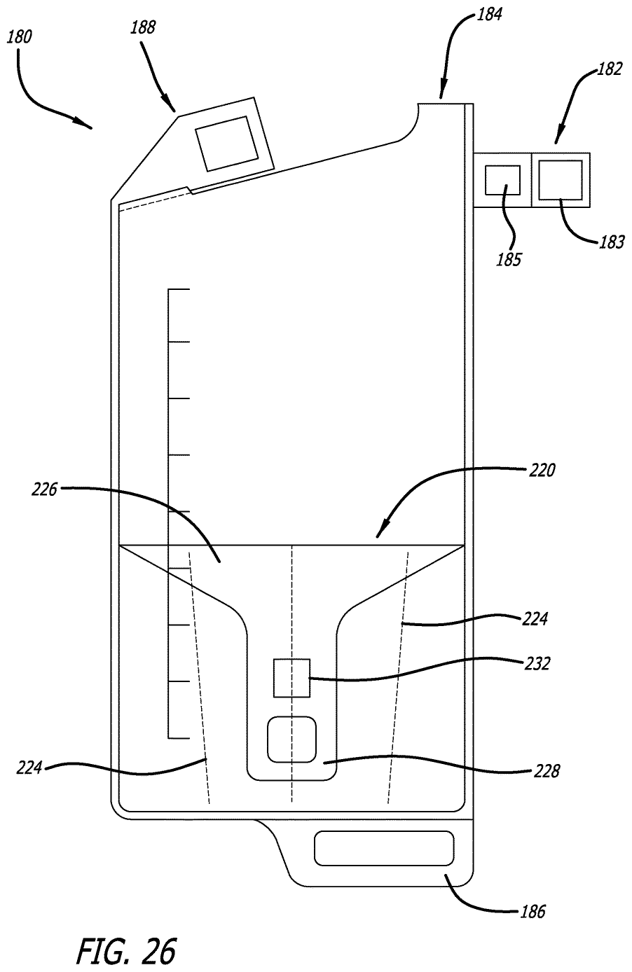

FIGS. 26, 40, and 41 illustrate various disposal means for disposing the drainage bag 180 in accordance with some embodiments.

As shown, the disposal means can be a disposal pocket 220 or a disposal pouch 222 respectively integrated in or removably attached to another longitudinal end portion of the drainage bag 180 opposite that including the urine inlet 184.

FIG. 26 provides an example of the disposal pocket 220 incorporated into an anterior face of the drainage bag 180 for folding the longitudinal end portion of the drainage bag 180 including the urine inlet 184 into the disposal pocket 220 for a used state of the drainage bag 180. However, the disposal pocket 220 can alternatively be incorporated into a posterior face of the drainage bag 180 for folding the longitudinal end portion of the drainage bag 180 including the urine inlet 184 into the disposal pocket 220. Optionally, the disposal pocket can be pleated with pleats 224 or otherwise include additional polymeric material of the drainage bag 180 for expanding the disposal pocket 220 to accommodate the longitudinal end portion of the drainage bag 180 including the urine inlet 184 therein. Further, the disposal pocket 220 can include a fold-over extension 226 formed of the polymeric material of the disposal pocket for sealing the longitudinal end portion of the drainage bag 180 including the urine inlet 184 in the disposal pocket 220 for a substantially or entirely leak-free used state of the drainage bag 180. Notably, the fold-over extension 226 of the disposal pocket 220 can include a handle 228 to facilitate folding the fold-over extension 226 over the posterior face of the drainage bag 180, if the disposal pocket 220 is incorporated into the anterior face of the drainage bag 180, or the anterior face of the drainage bag 180, if the disposal pocket 220 is incorporated into the posterior face of the drainage bag 180. Further, the fold-over extension 226 of the disposal pocket 220 can include an adhesive strip or patch 232 configured to seal the fold-over extension 226 over the posterior or anterior face of the drainage bag 180 for the substantially or entirely leak-free used state of the drainage bag 180.

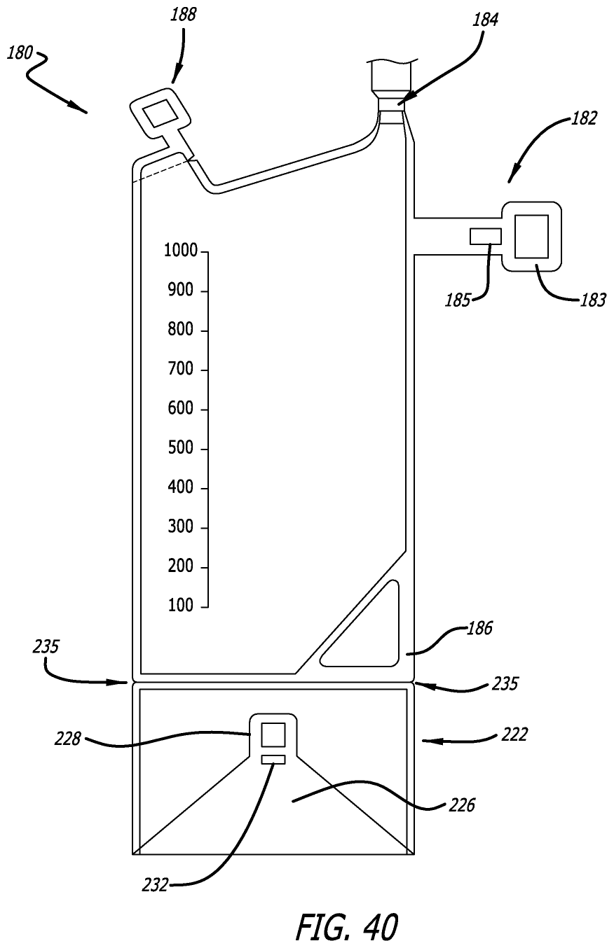

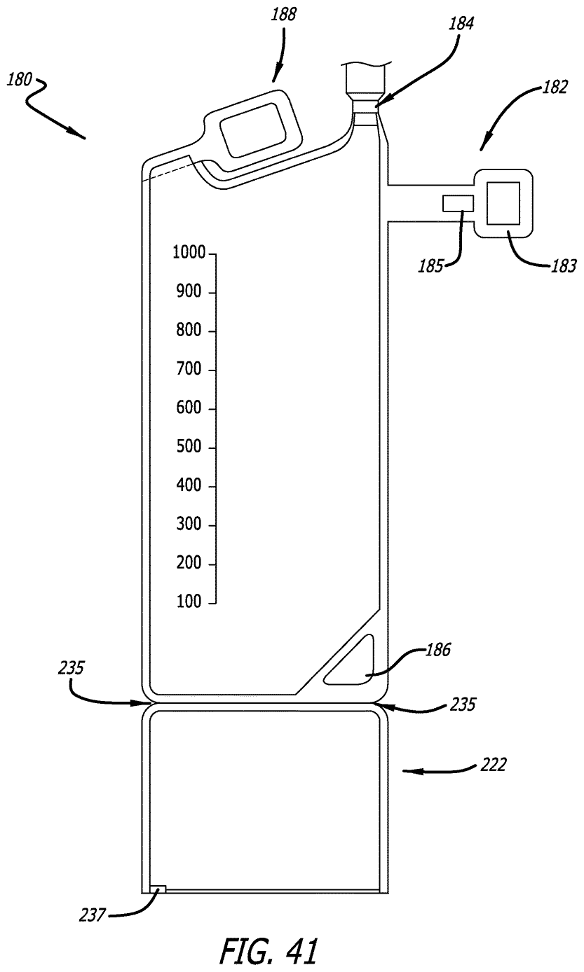

FIGS. 40 and 41 provides examples of the disposal pouch 222 removably attached to the other longitudinal end portion of the drainage bag 180 opposite that including the urine inlet 184 for enclosing the drainage bag 180 in the disposal pouch 222 for a used state of the drainage bag 180. Like the tear tab 188 for creating the urine outlet, the drainage bag 180 can be configured for pulling the disposal pouch 222 directly across a weakened (e.g., scored, perforated, etc.) separation line between the disposal pouch 222 and a remainder or main portion of the drainage bag 180 starting from one of two notches 235 to separate the disposal pouch 222 from the remainder or main portion of the drainage bag 180. As shown in FIG. 40, the disposal pouch 222 can include the fold-over extension 226 formed of the polymeric material of the drainage bag 180 for sealing the drainage bag 180 in the disposal pouch 222 after enclosing the remainder or main portion of the drainage bag 180 therein for a substantially or entirely leak-free used state of the drainage bag 180. Notably, the fold-over extension 226 of the disposal pouch 222 can include the handle 228 to facilitate folding the fold-over extension 226 over an opposite face of the drainage bag 180. Further, the fold-over extension 226 of the disposal pouch 222 can include the adhesive strip or patch 232 configured to seal the fold-over extension 226 over the opposite face of the drainage bag 180 for the substantially or entirely leak-free used state of the drainage bag 180. As shown in FIG. 41, the disposal pouch 222 can alternatively include a zipper 237 instead of the fold-over extension 226 for sealing the drainage bag 180 in the disposal pouch 222 after enclosing the remainder or main portion of the drainage bag 180 therein for the substantially or entirely leak-free used state of the drainage bag 180.

FIGS. 1, 3, 42-46, and 61-64 illustrate various rigid carrying cases 150, 250, 350, and 650 of the intermittent-catheter system 100 in accordance with some embodiments. Notably, such carrying cases 150, 250, 350, and 650 can be reusable such that the intermittent-catheter system 100 can be completed or made whole again after use with one or more supplemental packages, each of which includes at least the drainage assembly 170 or the drainage components thereof.

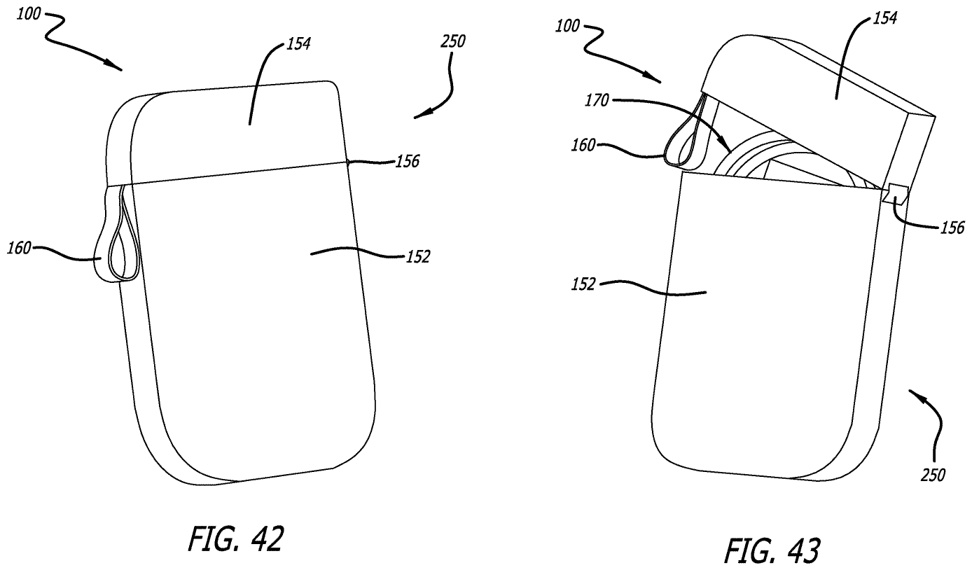

As to the carrying cases 150 and 250 of FIGS. 1, 3, 42, and 43, each carrying case of the carrying cases 150 and 250 includes a body 152 and a covering thereover such as a cap 154 for enclosing therein at least the drainage assembly 170 or the drainage components thereof in an unused state. While not shown, it should be understood that the intermittent-catheter system 100 can further include a covering over the body 152 akin to that set forth below for the tray packages 1050, 1150, and 1250 in the intermittent-catheter system 500; however, such a covering should be considered tamper-proof packaging for showing the intermittent-catheter system 100 remains in an unused state when the tamper-proof packaging remains intact. As shown, the body 152 and the cap 154 can be connected to each other by one or more hinges 156 such as one or more living hinges, one or more self-closing spring hinges, or the like on a minor longitudinal side of each carrying case of the carrying cases 150 and 250, independently. Further, each carrying case of the carrying cases 150 and 250 can independently include a clip 158, a rigid extension from the cap 154 such as a tab or a handle with a through hole, a pull strap 160, or the like on its minor longitudinal side opposite that including the one-or-more hinges 156. In an example, the carrying case 150 can include the one-or-more living hinges on one minor longitudinal side of the carrying case 150 and the clip 158 on the other minor longitudinal side of the carrying case 150 as shown in FIGS. 1 and 3. In another example, the carrying case 250 can include the one-or-more self-closing spring hinges on one minor longitudinal side of the carrying case 250 and the pull strap 160 on the other minor longitudinal side of the carrying case 250 as shown in FIGS. 42 and 43. In addition, each carrying case of the carrying cases 150 and 250 can independently include a gasket 162 between the body 152 and the cap 154, the carrying case 150 or 250, as the case might be, thereby configured to provide enhanced secondary containment for any residual urine that leaks from the intermittent-catheter assembly 110 in the used state of the intermittent-catheter assembly 110. Indeed, with such a configuration, the carrying case 150 or 250 is further configured for enclosing therein at least the intermittent-catheter assembly 110 in a used state of the intermittent-catheter assembly 110.

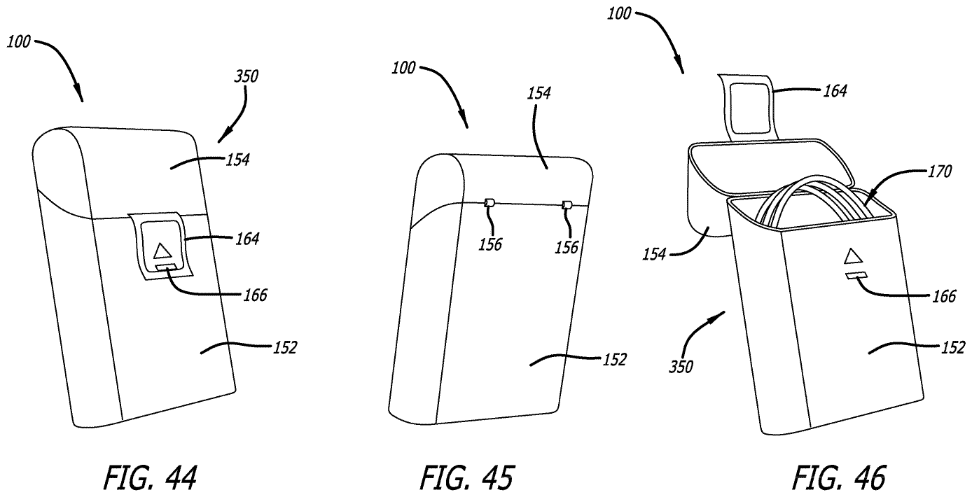

As to the carrying case 350 of FIGS. 44-46, the carrying case 350 also includes the body 152 and the covering thereover such as the cap 154 for enclosing therein at least the drainage assembly 170 or the drainage components thereof in an unused state. As shown, the body 152 and the cap 154 can be connected to each other by the one-or-more hinges 156 on a major longitudinal side of the carrying case 350, which is different than the carrying case 150 or 250. In additional contrast to the carrying case 150 or 250, the anterior face of the body 152 of the carrying case 350 can have a shorter length than the posterior face of the body 152, thereby providing a larger opening in the body 152 for greater access to components of the intermittent-catheter system 100 within the carrying case 350. Complementarily, the anterior face of the cap 154 of the carrying case 350 can have a longer length than the posterior face thereof. Further, the carrying case 350 can include a catch 164 on the major longitudinal side of the carrying case 350 opposite that including the one-or-more hinges 156. Indeed, the catch 164 can extend from the anterior face of the cap 154 such that it extends over the anterior face of the body 152 of the carrying case 350 where it can catch a keeper 166 in the anterior face of the carrying case 350 for fastening the cap 154 and body 152 together as shown in FIG. 44. While not shown, the carrying case 350 can also include the gasket 162 between the body 152 and the cap 154 if the cap 154 is not formed of a rubberized material. Regardless, the carrying case 350 is configured to provide secondary containment for any residual urine that leaks from the intermittent-catheter assembly 110 in the used state of the intermittent-catheter assembly 110. Indeed, with such a configuration, the carrying case 350 is further for enclosing therein at least the intermittent-catheter assembly 110 in a used state of the intermittent-catheter assembly 110.

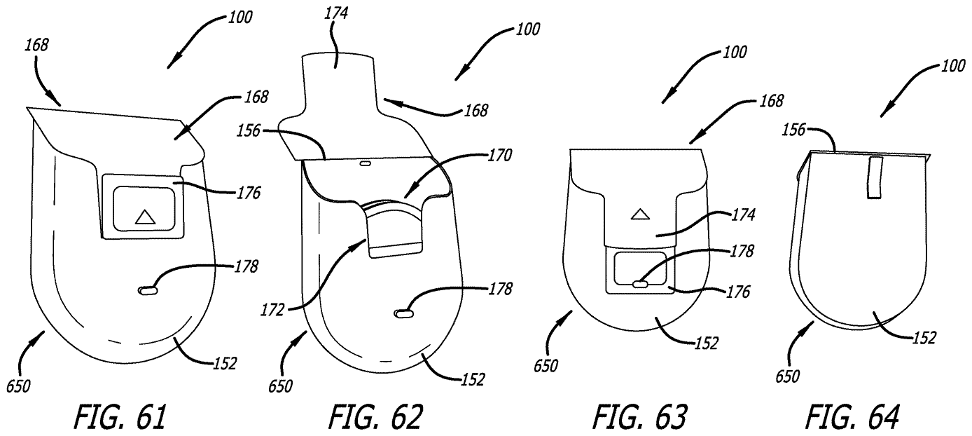

As to the carrying case 650 of FIGS. 61-64, the carrying case 650 also includes the body 152 and a covering thereover such as a lid 168 for enclosing therein at least the drainage assembly 170 or the drainage components thereof in an unused state. As shown, the body 152 and the lid 168 can be connected to each other by the one-or-more hinges 156 on a major longitudinal side of the carrying case 650. Like the carrying case 350, the anterior face of the body 152 of the carrying case 650 can have a shorter length than the posterior face of the body 152, thereby providing a larger opening in the body 152 for greater access to components of the intermittent-catheter system 100 within the carrying case 650. However, as shown in FIG. 62, the carrying case 650 can also include a cutout 172 in the anterior face connected the foregoing opening, thereby providing an even larger opening in the body 152 for even greater access to components of the intermittent-catheter system 100 within the carrying case 650. Complementarily, the anterior face of the lid of the carrying case 650 can be shaped with an extension 174 to overlap the opening and the cutout 172 thereof in the body 152 of the carrying case 650. Further, the carrying case 650 can include a hinged catch 176 on the major longitudinal side of the carrying case 650 opposite that including the one-or-more hinges 156, which hinged catch 176 can be used to indicate an unused state of the intermittent-catheter system 100 or a used state of the intermittent-catheter system 100. Indeed, in the unused state of the intermittent-catheter system 100, the hinged catch 176 can be squarely over the extension 174 of the lid 168 as shown in FIG. 61. In the used state of the intermittent-catheter system 100, the hinged catch 176 can extend from the extension 174 of the lid 168 over the anterior face of the body 152 of the carrying case 650 where it can catch a keeper 178 in the anterior face of the carrying case 650 for fastening the cap 154 and body 152 together as shown in FIG. 63. While not shown, the carrying case 650 can also include the gasket 162 between the body 152 and the cap 154 if the cap 154 is not formed of a rubberized material. Regardless, the carrying case 650 is configured to provide secondary containment for any residual urine that leaks from the intermittent-catheter assembly 110 in the used state of the intermittent-catheter assembly 110. Indeed, with such a configuration, the carrying case 650 is further for enclosing therein at least the intermittent-catheter assembly 110 in a used state of the intermittent-catheter assembly 110.

Notably, rigid packaging such as that of the carrying cases 150, 250, 350, and 650 offers better protection than soft packaging when the intermittent-catheter system 100 or the like is independently carried or otherwise toted; however, such rigid packaging can be more difficult than soft packaging to pack into luggage or the like, particularly when tightly packing such luggage.

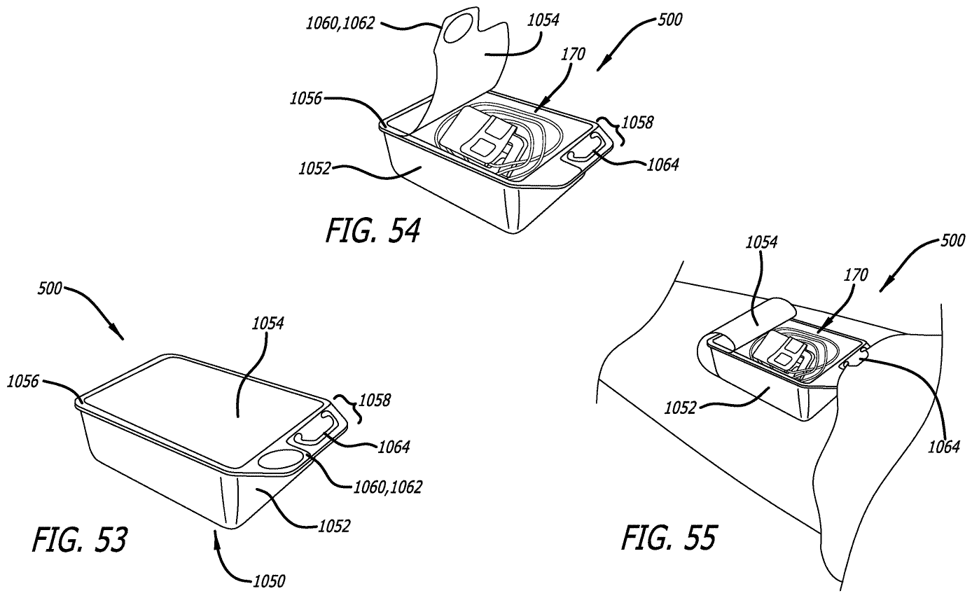

FIGS. 53-60 illustrate an intermittent-catheter system 500 in accordance with some embodiments. Notably, FIGS. 53, 56, 58, and 59 illustrate the intermittent-catheter system 500 in an unused state of the intermittent-catheter system 500, and FIGS. 54, 55, 57, and 60 illustrate the intermittent-catheter system 500 in a used state of the intermittent-catheter system 500.

As shown, the intermittent-catheter system 500 can include the drainage assembly 170 or the drainage components thereof like the intermittent-catheter system 100 set forth above. However, instead of the rigid carrying case 150, 250, 350, or 650 of the intermittent-catheter system 100, the intermittent-catheter system 500 can include a rigid tray package (or rigid tray packaging) selected from at least tray packages (or tray packaging) 1050, 1150, and 1250 set forth below.

As to the tray package 1050 of FIGS. 53-55, the tray package 1050 includes a tray 1052 having a rectangular shape, as defined by a longitudinal cross section of the tray 1052, a covering 1054 over an otherwise open face of the tray 1052 for enclosing therein at least the drainage assembly 170 or the drainage components thereof in an unused state, and a securing mechanism for securing the intermittent-catheter system 500 during a catheterization procedure. As shown, the tray 1052 can include a lip 1056 around the tray 1502 and a lip extension 1058 extending from the lip 1056 in a longitudinal end portion of the tray 1052. The covering 1054 can be sealed to the tray 1052 by way of a resealable seal around the lip 1056 of the tray 1052; however, a covering extension 1060 of the covering 1054 extending over the lip extension 1058 of the tray 1052 is notably not sealed to the lip extension 1058. Indeed, such a covering extension 1060 of the covering 1054, which can be a tab or handle 1062, as shown, is advantageously not sealed to the lip extension 1058 of the tray 1052 to facilitate grasping the covering extension 1060 and at least partially peeling the covering 1054 from the tray 1052 thereby for access to the components of the intermittent-catheter system 500 within the tray package 1050. When the covering extension 1060 of the covering 1054 is the ring-shaped handle 1062 or the like as shown in FIGS. 53 and 54, the rigid but flexible lip extension 1058 of the tray 1052 can be bent toward a base of the tray 1052 with a finger through the hole of the handle 1062 to separate the handle 1062 from the lip extension 1058 for grasping the handle 1062. Further, the lip extension 1058 of the tray 1052 can include an integrated clip 1064 for the securing mechanism, the integrated clip 1064 for conveniently clipping the tray 1052 and, thus, the intermittent-catheter system 500 to a user's shirt during a catheterization procedure. Lastly, being that the covering 1054 can be resealed to the tray 1052 by way of the resealable seal around the lip 1056 of the tray 1052, the tray package 1050 is thereby configured to provide secondary containment for any residual urine that leaks from the intermittent-catheter assembly 110 in the used state of the intermittent-catheter assembly 110. Indeed, with such a configuration, the tray package 1050 is further configured for enclosing therein at least the intermittent-catheter assembly 110 in a used state of the intermittent-catheter assembly 110.

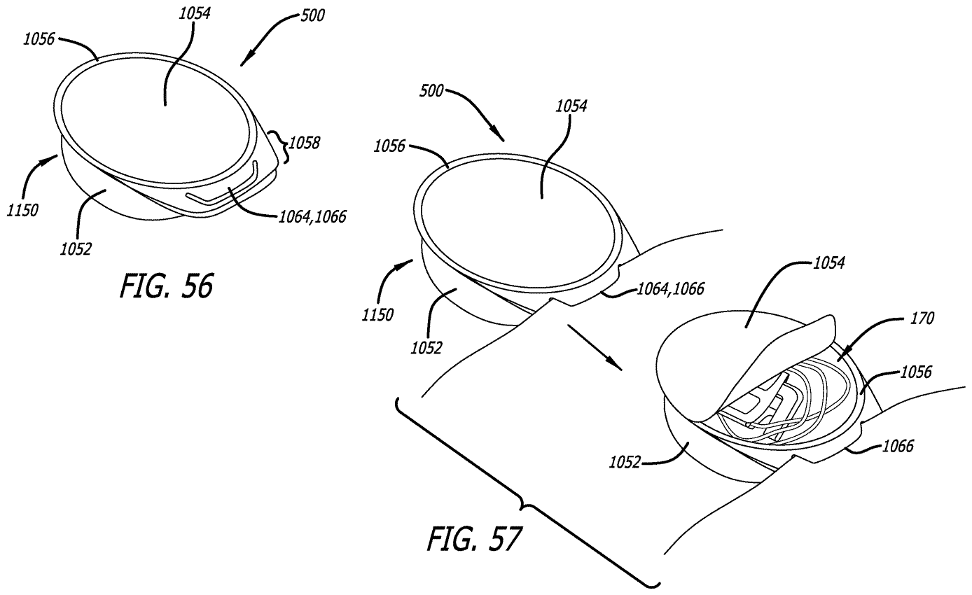

As to the tray package 1150 of FIGS. 56 and 57, the tray package 1150 includes the tray 1052 having an elliptical shape (e.g., a circular tray or an oval tray), as defined by a longitudinal cross section of the tray 1052, the covering 1054 over an otherwise open face of the tray 1052 for enclosing therein at least the drainage assembly 170 or the drainage components thereof in an unused state, and the securing mechanism for securing the intermittent-catheter system 500 during a catheterization procedure. Like the rectangular tray 1052, the elliptical tray 1052 can include the lip 1056 around the tray 1052 and the lip extension 1058 extending from the lip 1056 of the tray 1052, which lip extension 1058 can also include the integrated clip 1064 for conveniently clipping the tray 1052 and, thus, the intermittent-catheter system 500 to a user's shirt during a catheterization procedure. Differently, however, the clip 1064 of the elliptical tray 1052 is advantageously centered in the lip extension 1058 of the tray 1052 instead of off to one side thereof as in the rectangular tray 1052. The covering 1054 can be sealed to the tray 1052 by way of a resealable seal around the lip 1056 of the tray 1052. And while not shown, the covering 1054 of the tray package 1150 can include an unsealed covering extension akin to that of the covering extension 1060 of the tray package 1050; however, when the tray package 1150 includes such a covering extension, it extends over at least a tongue 1066 of the clip 1064 in the lip extension 1058 of the tray 1052. Whether the covering extension 1060 of the covering 1054 is a tab or a handle such as the ring-shaped handle 1062 shown in FIGS. 53 and 54, the rigid but flexible tongue 1066 of the clip 1064 can be bent toward a base of the tray 1052 with a finger to separate the covering extension 1060 from the lip extension 1058 to facilitate grasping the covering extension 1060 and at least partially peeling the covering 1054 from the tray 1052 thereby for access to the components of the intermittent-catheter system 500 within the tray package 1150. Lastly, being that the covering 1054 can be resealed to the tray 1052 by way of the resealable seal around the lip 1056 of the tray 1052, the tray package 1150 is thereby configured to provide secondary containment for any residual urine that leaks from the intermittent-catheter assembly 110 in the used state of the intermittent-catheter assembly 110. Indeed, with such a configuration, the tray package 1150 is further configured for enclosing therein at least the intermittent-catheter assembly 110 in a used state of the intermittent-catheter assembly 110.

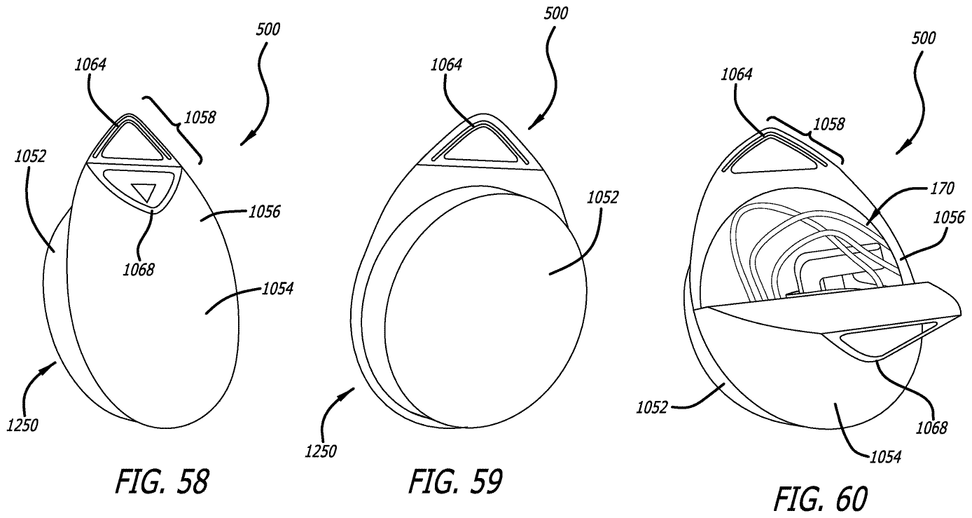

As to the tray package 1250 of FIGS. 58-60, the tray package 1250 also includes the tray 1052 having the elliptical shape (e.g., a circular tray or an oval tray), as defined by a longitudinal cross section of the tray 1052, but, differently, the lip 1056 around the tray 1052 has a teardrop shape. In addition, the tray package 1250 includes the covering 1054 over an otherwise open face of the tray 1052 for enclosing therein at least the drainage assembly 170 or the drainage components thereof in an unused state, as well as and the securing mechanism for securing the intermittent-catheter system 500 during a catheterization procedure. The lip 1056 around the tray 1052 includes the lip extension 1058 extending from the lip 1056 of the tray 1052, but the lip extension 1058 comes to a point, thereby giving the teardrop-shaped lip 1056 its shape. The pointed lip extension 1058 can include the integrated clip 1064 for conveniently clipping the tray 1052 and, thus, the intermittent-catheter system 500 to a user's shirt during a catheterization procedure. Notably, the clip 1064 of the tray 1052 is pointed, and such a pointed clip 1064 can have a deeper bite and, therefore, a stronger grip on the user's shirt than the clip 1064 of the tray package 1050 or 1150 on account of a length of the pointed lip extension 1058. In addition, the pointed clip 1064 can be made of a different, stronger material than a remainder of the tray 1052 such as a metal clip overmolded into a polymeric tray, thereby further strengthening the grip of the pointed clip 1064. Lastly, the pointed clip 1064 of the tray 1052 is advantageously centered in the pointed lip extension 1058 of the tray 1052 instead of off to one side thereof as in the rectangular tray 1052. The covering 1054 can be sealed to the tray 1052 by way of a resealable seal around the lip 1056 of the tray 1052. And while the covering 1054 can include an unsealed covering extension akin to that of the covering extension 1060 of the tray package 1050 that extends over the pointed lip extension 1058, the tray package 1250 of FIG. 58 is instead shown with a triangular handle 1068 oriented opposite to the pointed lip extension 1058 to facilitate grasping the handle 1068 and at least partially peeling the covering 1054 from the tray 1052 thereby for access to the components of the intermittent-catheter system 500 within the tray package 1250. Lastly, being that the covering 1054 can be resealed to the tray 1052 by way of the resealable seal around the lip 1056 of the tray 1052, the tray package 1250 is thereby configured to provide secondary containment for any residual urine that leaks from the intermittent-catheter assembly 110 in the used state of the intermittent-catheter assembly 110. Indeed, with such a configuration, the tray package 1250 is further configured for enclosing therein at least the intermittent-catheter assembly 110 in a used state of the intermittent-catheter assembly 110.

Notably, rigid packaging such as that of the tray packages 1050, 1150, and 1250 offers better protection than soft packaging when the intermittent-catheter system 500 or the like is independently carried or otherwise toted; however, such rigid packaging can be more difficult than soft packaging to pack into luggage or the like, particularly when tightly packing such luggage.

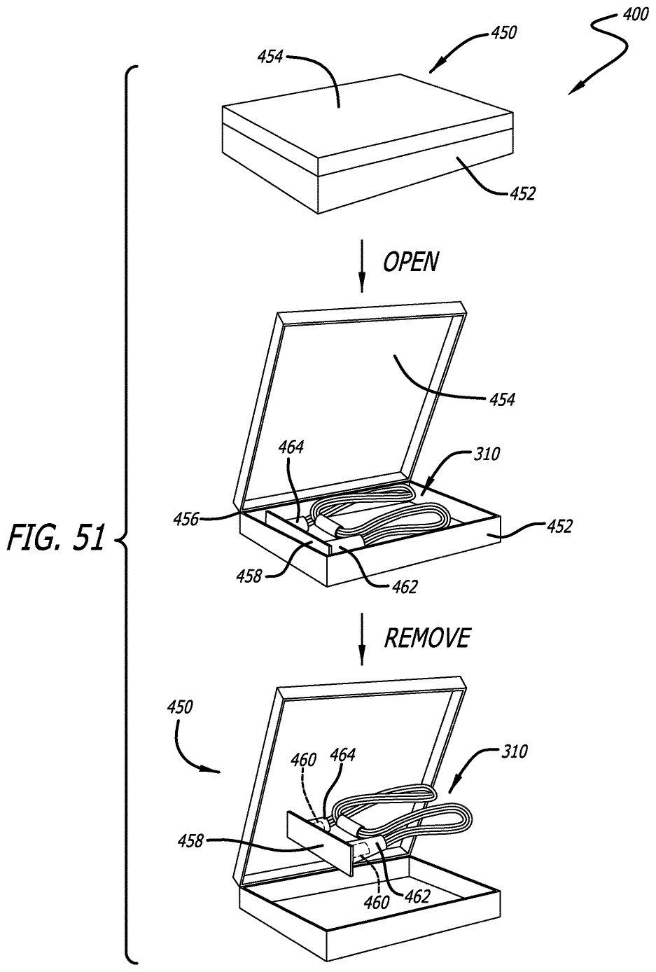

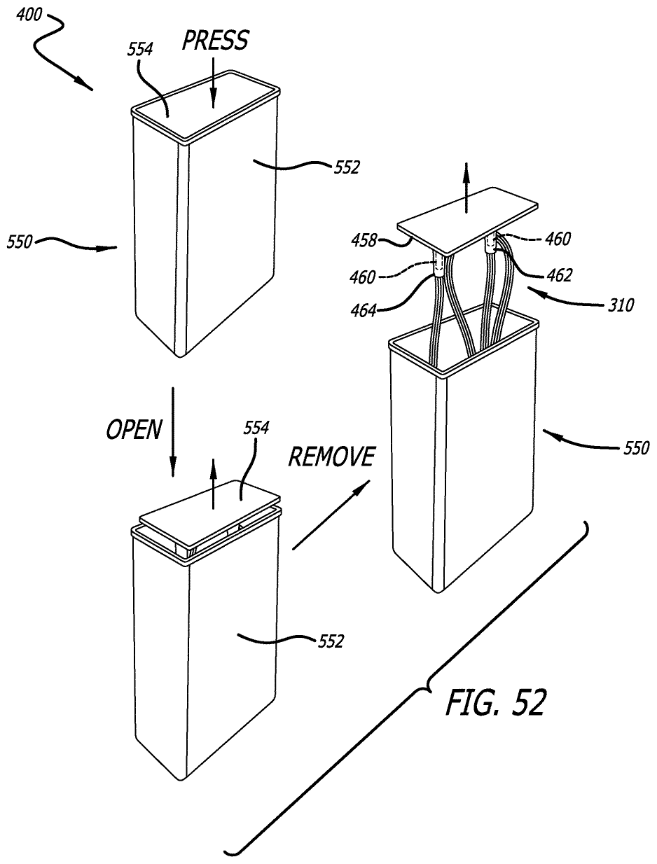

FIGS. 51 and 52 illustrate an intermittent-catheter system 400 in accordance with some embodiments. Notably, FIGS. 51 and 52 illustrate the intermittent-catheter system 400 in accordance with an opening mechanism for opening the rigid carrying case 450 or 550 of the intermittent-catheter system 400 and, thereby, transitioning the intermittent-catheter system 400 from a storage state to an opened state thereof or vice versa.

As shown, the intermittent-catheter system 400 can include the drainage assembly 170 or the drainage components thereof like the intermittent-catheter system 100 set forth above. However, instead of the rigid carrying case 150, 250, 350, or 650 of the intermittent-catheter system 100, which passively encloses the drainage assembly 170 or the drainage components thereof in their unused or used states, the carrying case 450 and 550 interact with at least the intermittent-catheter assembly 310.

As to the carrying case 450 of FIG. 51, the carrying case 450 includes a body 452 and a covering thereover such as a lid 454 for enclosing therein at least the drainage assembly 170 or the drainage components thereof in an unused state. As shown, the body 452 and the lid 454 can be connected to each other by a longitudinal living hinge 456 on a major longitudinal side of the carrying case 450, the body 452 and the lid 454 thereby providing a clamshell-type opening for unencumbered access to components of the intermittent-catheter system 500 for removal from the carrying case 450 as shown. Further, the carrying case 450 can include a storage plate 458 having a suitably sized pair of posts 460 extending therefrom configured for mounting an intermittent-catheter assembly 310 thereon in either an unused state of the intermittent-catheter assembly 310 or a used state of the intermittent-catheter assembly 310. Indeed, a funnel 462 of the intermittent-catheter assembly 310 can be mounted on a first post of the pair of posts 460 extending from the storage plate 458, and an end cap 464 of a sterility-preserving enclosure 466 of the intermittent-catheter assembly 310 can be mounted on a second post of the pair of posts 460. As such, the storage plate 458 and the pair of posts 460 thereof can protect the catheter tube 134 from contamination as well as keep any lubricious coating 135 on the catheter tube 134 or any lubricant around the catheter tube 134 contained within the sterility-preserving enclosure 466 or storage sheath 144 thereof in the unused state of the intermittent-catheter assembly 310. Further, the storage plate 458 and the pair of posts 460 thereof can keep any residual urine in or around the catheter tube 134 contained within the urine-retaining enclosure or storage sheath 144 thereof in the used state of the intermittent-catheter assembly 310. The carrying case 450, in turn, can be configured with a gasket like the gasket 162 set forth above or the like, to provide enhanced secondary containment, particularly for any residual urine that could leak from the intermittent-catheter assembly 310 in the used state of the intermittent-catheter assembly 310.

As to the carrying case 550 of FIG. 52, the carrying case 550 includes a body 552 and a covering thereover such as a lid 554 for enclosing therein at least the drainage assembly 170 or the drainage components thereof in an unused state. As shown, the body 552 and the lid 554 can be operably connected to each other by a low-dexterity friendly press-to-open lid-opening mechanism (e.g., a cam-and-follower mechanism) in a longitudinal end of the carrying case 550, wherein the lid 554 is configured to separate from the body 552 when an external face of the lid 554 is pressed into the body 552, thereby providing an opening for access to components of the intermittent-catheter system 500 for removal from the carrying case 550 as shown. Further, an internal face of the lid 554 can be configured as the storage plate 458 having the suitably sized pair of posts 460 extending therefrom configured for mounting the intermittent-catheter assembly 310 thereon in either an unused state of the intermittent-catheter assembly 310 or a used state of the intermittent-catheter assembly 310. Indeed, as set forth above, the funnel 462 of the intermittent-catheter assembly 310 can be mounted on the first post of the pair of posts 460 extending from the storage plate 458, and the end cap 464 of the intermittent-catheter assembly 310 can be mounted on the second post of the pair of posts 460. As such, the storage plate 458 and the pair of posts 460 thereof can protect the catheter tube 134 from contamination as well as keep any lubricious coating 135 on the catheter tube 134 or any lubricant around the catheter tube 134 contained within the sterility-preserving enclosure 466 or storage sheath 144 thereof in the unused state of the intermittent-catheter assembly 310. Further, the storage plate 458 and the pair of posts 460 thereof can keep any residual urine in or around the catheter tube 134 contained within the urine-retaining enclosure or storage sheath 144 thereof in the used state of the intermittent-catheter assembly 310. The carrying case 550, in turn, can be configured with a gasket like the gasket 162 set forth above or the like, to provide enhanced secondary containment, particularly for any residual urine that could leak from the intermittent-catheter assembly 310 in the used state of the intermittent-catheter assembly 310.

Notably, rigid packaging such as that of the carrying case 450 or 550 offers better protection than soft packaging when the intermittent-catheter system 500 or the like is independently carried or otherwise toted; however, such rigid packaging can be more difficult than soft packaging to pack into luggage or the like, particularly when tightly packing such luggage.

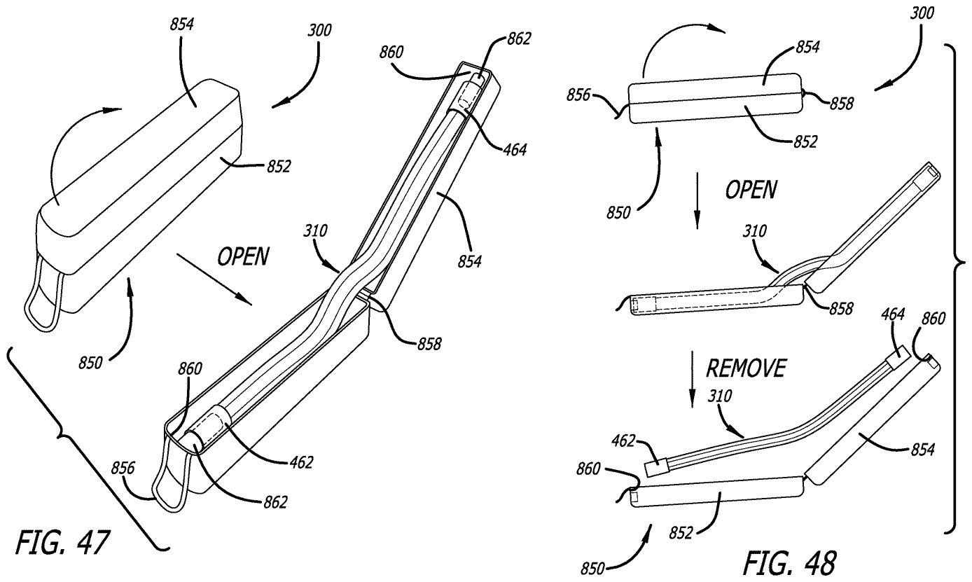

FIGS. 47-50 illustrate an intermittent-catheter system 300 in accordance with some embodiments. Notably, each figure of FIGS. 47-50 illustrates the intermittent-catheter system 300 in accordance with an opening mechanism for opening the rigid catheter housing 850 or 950 of the intermittent-catheter system 300 and, thereby, transitioning the intermittent-catheter system 300 from a storage state to an opened state thereof or vice versa.

As shown, the intermittent-catheter system 300 can include the intermittent-catheter assembly 310 set forth above, notably, without the drainage bag 180 or other bulky drainage components. Further, instead of the rigid carrying case 150, 250, 350, or 650 of the intermittent-catheter system 100, which passively encloses at least the intermittent-catheter assembly 310 in its unused or used states, the catheter housings 850 and 950 interact with at least the intermittent-catheter assembly 310.

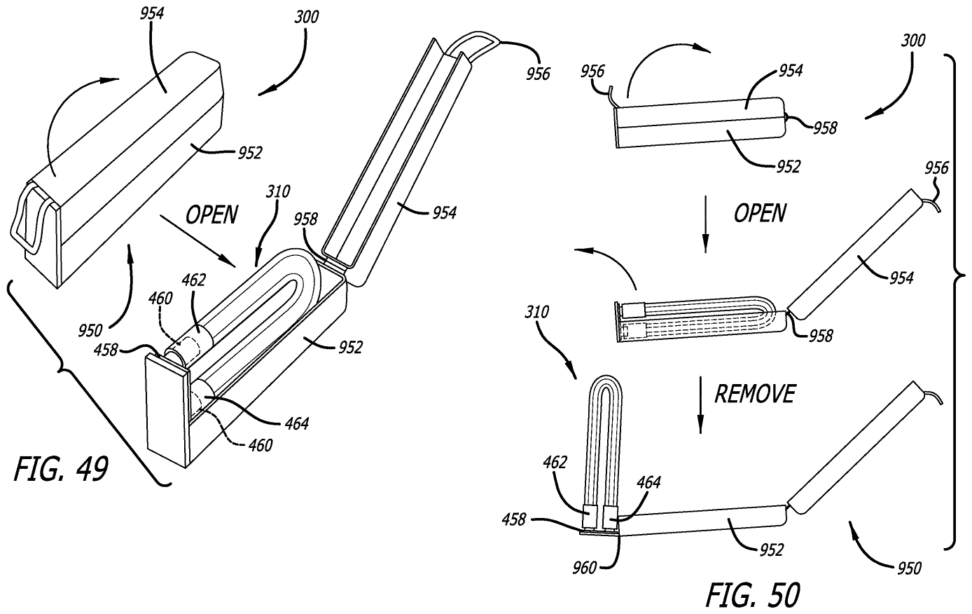

Beginning with the catheter housing 950 of FIGS. 49 and 50, the catheter housing 950 includes a body 952 and a covering thereover such as a lid 954 with an optional opening loop 956 or the like, the catheter housing 950 for enclosing therein at least the intermittent-catheter assembly 310 in the unused state. As shown, the body 952 and the lid 954 can be connected to each other by a living hinge 958 in a longitudinal end portion of the catheter housing 950, the body 952 and the lid 954 thereby providing a clapperboard-type opening for unencumbered access to at least the intermittent-catheter assembly 310 for removal from the catheter housing 950 as shown. Further, the catheter housing 950 can include the storage plate 458, which can be configured to sit within a longitudinal end portion of the catheter housing 950 opposite the living hinge 958 or integrated into an internal face of a minor side of the catheter housing 950 opposite the living hinge 958. Notably, the minor side of the catheter housing 950 including the storage plate 458 can also be connected to the body 952 by a living hinge 960 such that the minor side of the catheter housing 950 can be folded away from a remainder of the body 952 such as folded down onto an external surface for low-dexterity friendly removal of the intermittent-catheter assembly 310 from the catheter housing 950. Again, the storage plate 458 can have the suitably sized pair of posts 460 extending therefrom configured for mounting the intermittent-catheter assembly 310 thereon in either an unused state of the intermittent-catheter assembly 310 or a used state of the intermittent-catheter assembly 310. Indeed, the funnel 462 of the intermittent-catheter assembly 310 can be mounted on the first post of the pair of posts 460 extending from the storage plate 458, and the end cap 464 of the intermittent-catheter assembly 310 can be mounted on the second post of the pair of posts 460. As such, the storage plate 458 and the pair of posts 460 thereof can protect the catheter tube 134 from contamination as well as keep any lubricious coating 135 on the catheter tube 134 or any lubricant around the catheter tube 134 contained within the sterility-preserving enclosure 466 or storage sheath 144 thereof in the unused state of the intermittent-catheter assembly 310. Further, the storage plate 458 and the pair of posts 460 thereof can keep any residual urine in or around the catheter tube 134 contained within the urine-retaining enclosure or storage sheath 144 thereof in the used state of the intermittent-catheter assembly 310. The catheter housing 950, in turn, can be configured with a gasket like the gasket 162 set forth above or the like, to provide enhanced secondary containment, particularly for any residual urine that could leak from the intermittent-catheter assembly 310 in the used state of the intermittent-catheter assembly 310.

As to the catheter housing 850 of FIGS. 47 and 48, the catheter housing 850 includes a body 852 and a covering thereover such as a lid 854 with an optional opening loop 856 or the like, the catheter housing 850 for enclosing therein at least the intermittent-catheter assembly 310 in the unused state. As shown, the body 852 and the lid 854 can be connected to each other by a living hinge 858 in a longitudinal end portion of the catheter housing 850, the body 852 and the lid 854 thereby providing a clapperboard-type opening for unencumbered access to at least the intermittent-catheter assembly 310 for removal from the catheter housing 850 as shown. Further, the catheter housing 850 can include a split or two-piece storage plate 860 distributed between body and lid pieces of a longitudinal end portion of the catheter housing 850 opposite that including living hinge 858. Such a storage plate 860 can be integrated into a split internal face of a split or two-piece minor side of the catheter housing 850 opposite that including the living hinge 858. Each piece of the split or two-piece storage plate 860 can have a suitably sized post extending therefrom for a pair of posts 862 configured for mounting the intermittent-catheter assembly 310 thereon in either an unused state of the intermittent-catheter assembly 310 or a used state of the intermittent-catheter assembly 310. Indeed, the funnel 462 of the intermittent-catheter assembly 310 can be mounted on a first post of the pair of posts 862 extending from a first piece of the split or two-piece storage plate 860, and the end cap 464 of the intermittent-catheter assembly 310 can be mounted on a second post of the pair of posts 862 extending from a second piece of the split or two-piece storage plate 860. As such, the split or two-piece storage plate 860 and the pair of posts 862 thereof can protect the catheter tube 134 from contamination as well as keep any lubricious coating 135 on the catheter tube 134 or any lubricant around the catheter tube 134 contained within the sterility-preserving enclosure 466 or storage sheath 144 thereof in the unused state of the intermittent-catheter assembly 310. Notably, when opening the catheter housing 850 in accordance with the opening mechanism for opening the catheter housing 850, the split or two-piece storage plate 860 is separated such that the first and second pieces of the split or two-piece storage plate 860 oppose each other, thereby providing unencumbered access to the intermittent-catheter assembly 310 for removal from the catheter housing 850 for use or replacement in the catheter housing 850 after use. As to the latter, the split or two-piece storage plate 860 and the pair of posts 862 thereof can keep any residual urine in or around the catheter tube 134 contained within the urine-retaining enclosure or storage sheath 144 thereof in the used state of the intermittent-catheter assembly 310. The catheter housing 850, in turn, can be configured with a gasket like the gasket 162 set forth above or the like, to provide enhanced secondary containment, particularly for any residual urine that could leak from the intermittent-catheter assembly 310 in the used state of the intermittent-catheter assembly 310.

Notably, rigid packaging such as that of the catheter housing 850 or 950 offers better protection than soft packaging when the intermittent-catheter system 300 or the like is independently carried or otherwise toted; however, such rigid packaging can be more difficult than soft packaging to pack into luggage or the like, particularly when tightly packing such luggage.

### Methods of Male or Unisex Intermittent-Catheter Systems

As set forth below, methods can include methods of using the intermittent-catheter systems and assemblies disclosed herein; however, it should be understood that those methods can be further discerned from description of the intermittent-catheter systems and assemblies and how they are configured to function. Likewise, additional methods can be discerned from description of the intermittent-catheter systems and assemblies and how they are configured to function.

For example, a method of an intermittent-catheter system 100 can include removing the drainage assembly 170 in the unused state of the drainage assembly 170 from the rigid carrying case 150.

As set forth above, the drainage assembly can include the intermittent-catheter assembly 110 and the drainage bag 180.

Briefly, as to the intermittent-catheter assembly 110, the intermittent-catheter assembly 110 can include the intermittent catheter 130 and the sterility-preserving enclosure 132. The intermittent catheter 130 can include the catheter tube 134 and the funnel 136 over the proximal end portion of the catheter tube 134. The funnel 136 can have the funnel opening 142 for voiding urine. The sterility-preserving enclosure 132 can include the storage sheath 144 and the end cap 146. The storage sheath 144 and the end cap 146 can be configured to enclose at least the catheter tube 134 of the intermittent catheter 130 therein.

As to the drainage bag 180, the drainage bag 180 is optionally pre-connected to the funnel 136 of the intermittent catheter 130.

The method can also include inserting the distal end portion of the catheter tube 134 into a urethra; voiding urine from a bladder into the drainage bag 180 through the intermittent catheter 130; and enclosing at least the intermittent-catheter assembly 110 in the used state of the intermittent-catheter assembly 110 in the carrying case 150.

The method can also include inserting the end portion of the funnel 136 into the receptacle 148 of the distal portion of the end cap 146 to form the leak-free, ouroboros-type, end-to-end closure in the used state of the intermittent-catheter assembly 110. Alternatively, the method can include inserting the plug (not shown) of the distal portion of the end cap 146 into the funnel opening 142 of the funnel 136 to form the leak-free, ouroboros-type, end-to-end closure in the used state of the intermittent-catheter assembly 110. Regardless, the storage sheath 144 can extend between the end cap 146 and the funnel 136 of the intermittent catheter 130 in the used state of the intermittent-catheter assembly 110, thereby keeping any residual urine in or around the catheter tube 134 contained within the storage sheath 144 in the used state of the intermittent-catheter assembly 110.

The method can also include removing the strap 182 adhered to the folded portion of the drainage bag 180 maintained in the folded state of the drainage bag 180 by the strap 182. The strap 182 can extend from the longitudinal end portion of the drainage bag 180 including the urine inlet 184.

The method can also include unfolding the drainage bag 180. As set forth above, the folded state of the drainage bag 180 can include the one-or-more folds along the width such that the drainage bag 180 has a shorter length in the folded state than in the unfolded state of the drainage bag 180, the one-or-more folds along the length such that the drainage bag 180 has a shorter width in the folded state than in the unfolded state of the drainage bag 180, or some combination thereof.

The method can also include draining the urine from the drainage bag 180; and folding the longitudinal end portion of the drainage bag 180 including the urine inlet 184 into the disposal pocket 220 incorporated into the face of the drainage bag 180 for the used state of the drainage assembly 170. Notably, the disposal pocket 220 is in the other longitudinal end portion of the drainage bag 180 opposite that including the urine inlet 184. Alternatively, the method can include draining the urine from the drainage bag 180; removing the disposal pouch 222 attached to the other longitudinal end portion of the drainage bag 180 opposite that including the urine inlet 184; and enclosing the drainage bag 180 in the disposal pouch 222 for the used state of the drainage assembly 170.

### Female Intermittent-Catheter Systems

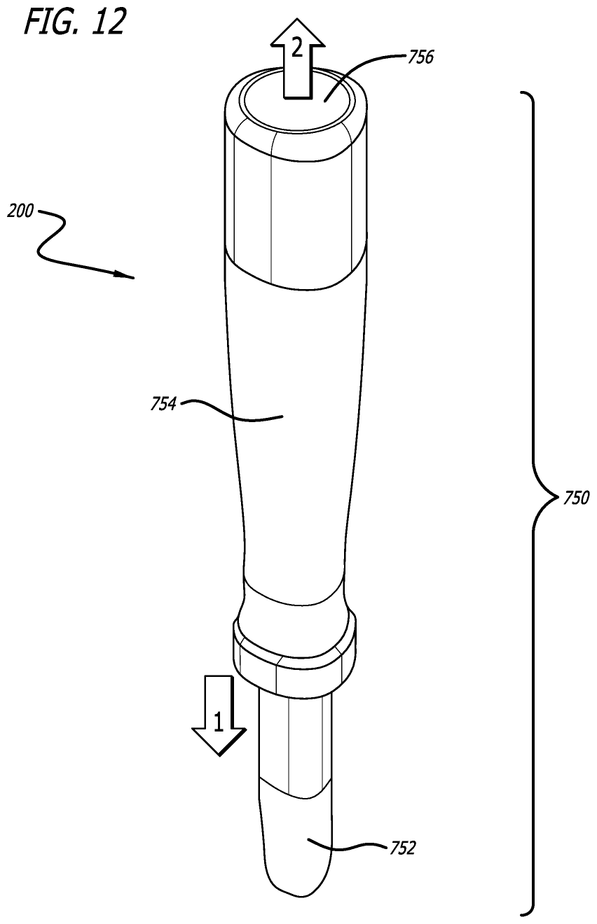

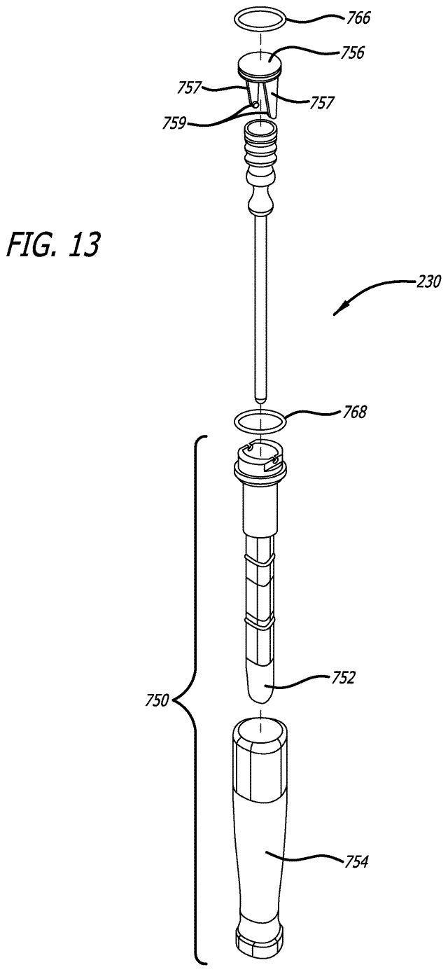

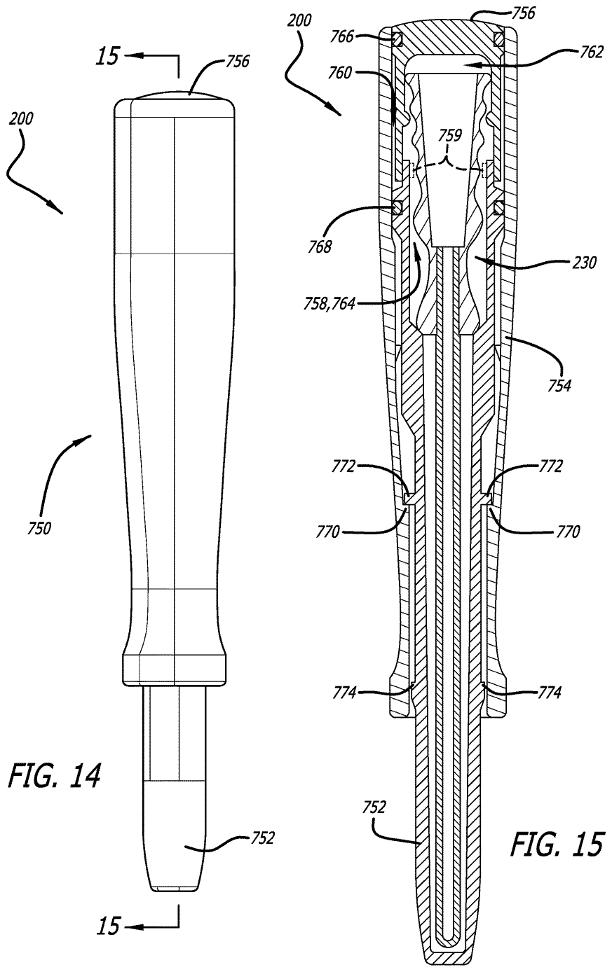

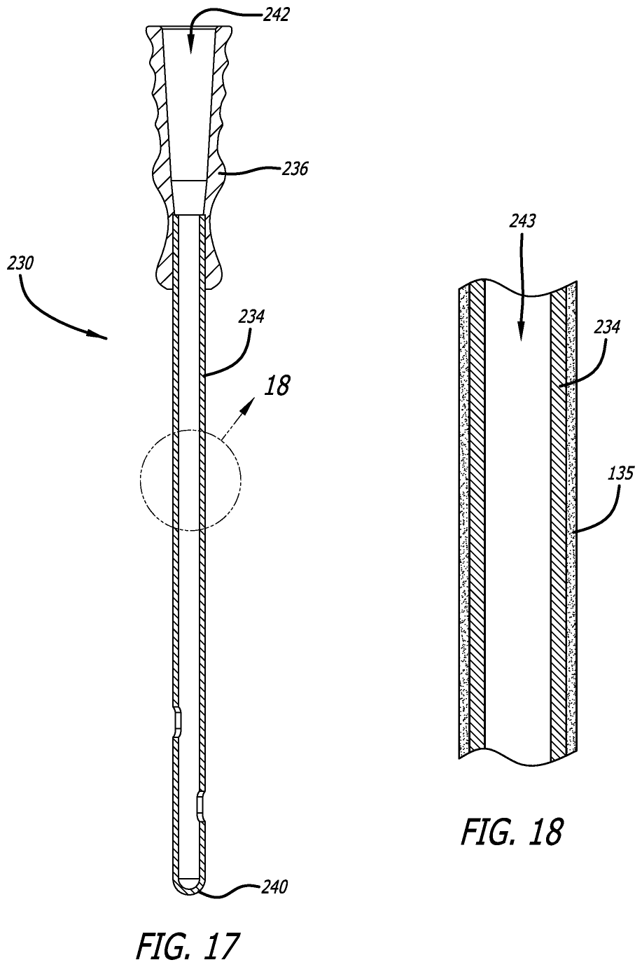

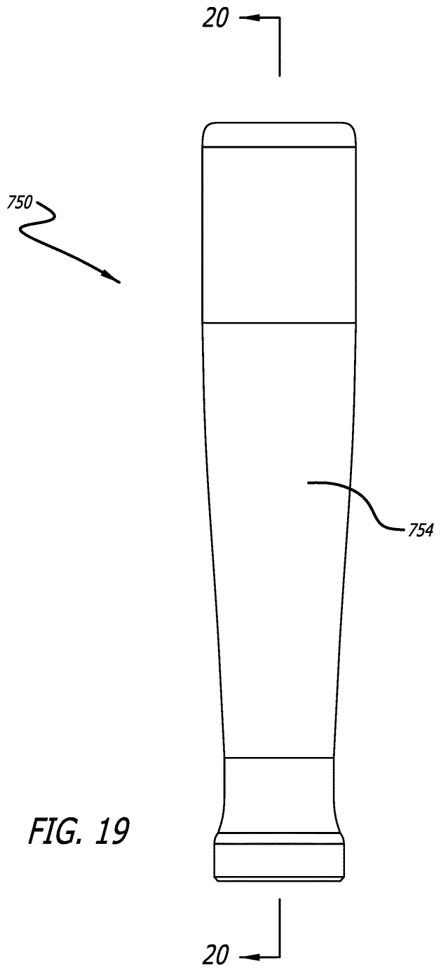

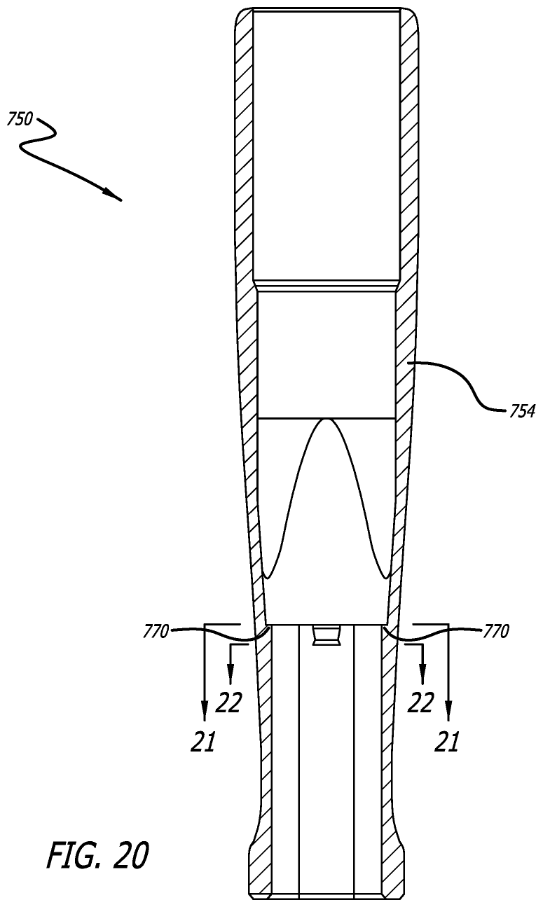

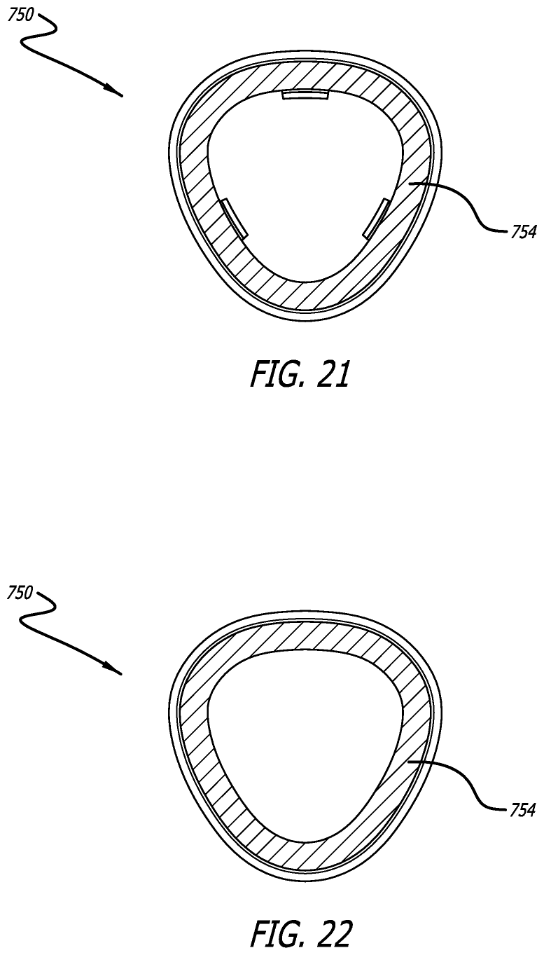

FIGS. 12-22 illustrate an intermittent-catheter system 200 or components thereof in accordance with some embodiments. Such an intermittent-catheter system 200 and its components are configured for use by female humans.

As shown, the intermittent-catheter system 200 can include an intermittent catheter 230 and a rigid catheter housing 750.

The intermittent-catheter system 200 can have a storage state and an opened state. In the storage state, the catheter housing 750 can be at a maximally extended state thereof with the intermittent catheter 230 sealed within the primary chamber 758 set forth below. In the opened state, the outer sleeve 754 of the catheter housing 750 is slid over the inner sleeve 752 of the catheter housing 750 such that the catheter housing 750 can be at a maximally collapsed state thereof with the end cap 756 popped out of the outer sleeve 754 and the funnel 236 of the intermittent catheter 230 exposed for removal of the intermittent catheter 230 from the catheter housing 750. However, it should be understood that the opened state of the intermittent-catheter system 200 can include the outer sleeve 754 of the catheter housing 750 slid over the inner sleeve 752 of the catheter housing 750 in an intermediate collapsed state thereof with the end cap 756 partially popped out of the outer sleeve 754 and the funnel 236 of the intermittent catheter 230 partially exposed.

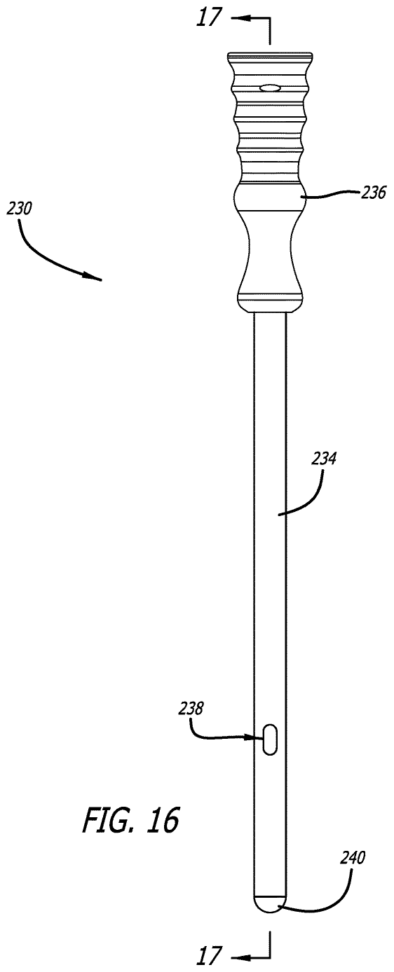

FIGS. 16-18 illustrate the intermittent catheter 230 in accordance with some embodiments.

The intermittent catheter 230 can include a catheter tube 234 and a funnel 236 over a proximal end portion of the catheter tube 234. The catheter tube 234 can include a plurality of eyelets 238 proximate a catheter tip 240, and the funnel 236 can have a funnel opening 242 in fluid communication with the eyelets 238 by way of a lumen 243 through the intermittent catheter 230 for voiding urine. In addition, the funnel 236 can include a plurality of ridges integrated into an outer surface of the funnel 236 configured for gripping the funnel 236 as a handle while removing the intermittent catheter 230 from the catheter housing 750 or voiding urine.

FIGS. 12-15 and 19-22 illustrate the catheter housing 750 in accordance with some embodiments.

The catheter housing 750 can include an inner sleeve 752, an outer sleeve 754 slidably mounted over the inner sleeve 752, and an end cap 756. As shown in FIG. 12, the catheter housing 750 is configured to collapse when the closed end portion of the inner sleeve 752 is held against a surface and the outer sleeve 754 is slid over the inner sleeve 752 toward the surface, thereby exposing the funnel 236 of the intermittent catheter 230 for removal of the intermittent catheter 230 from the catheter housing 750.

As shown, the end cap 756 can be a swivel cap including a pair of extension arms 757, wherein each extension arm of the pair of extension arms 757 includes a post 759 configured for insertion into a complementary through hole in an open-end portion of the inner sleeve 752. Notably, such an end cap 756 contributes to the following secondary chamber 760 but not the primary chamber 758. Alternatively, the end cap 756 can approximate a single-ended cylinder (e.g., a cylinder having a single closed end) that simultaneously contributes to both a primary chamber 758 and a secondary chamber 760. (See FIG. 15, wherein the post 759 of each extension arm of the pair of extension arms 757 is shown in dashed outline indicating its presence or absence in accordance with the swivel cap or the single-ended cylinder.) Indeed, when the end cap 756 approximates the single-ended cylinder, the end cap 756 can be configured to sit within an end portion of the outer sleeve 754 opposite a closed end portion of the inner sleeve 752 and simultaneously contribute to both the primary chamber 758 and the secondary chamber 760. The end cap 756 can overlap the open-end portion of the inner sleeve 752 in an interference fit such as a clearance fit to provide the primary chamber 758 between an end-cap cavity 762 of the end cap 756 and an inner-sleeve cavity 764 of the inner sleeve 752. Regardless of whether the end cap 756 is the swivel cap or that approximating the single-ended cylinder, an end-cap gasket 766 (e.g., a single end-cap gasket) around the end cap 756 and an inner-sleeve gasket 768 (e.g., a single inner-sleeve gasket) around the inner sleeve 752 can define longitudinal limits of the secondary chamber 760 between an interior surface of the outer sleeve 754 and exterior surfaces of the end cap 756 and the inner sleeve 752. Notably, the end-cap gasket 766 and the inner-sleeve gasket 768 provide sufficient friction for maintaining the storage state of the catheter housing 750 under typical usage scenarios, thereby averting inadvertent collapse into the opened state of catheter housing 750.

The primary chamber 758 and the secondary chamber 760 respectively provide primary containment and secondary containment for containing or otherwise keeping any lubricious coating 135 on the catheter tube 234 or any lubricant around the catheter tube 234 of the intermittent catheter 230, thereby preventing lubricant leakage from the catheter housing 750 in the storage state of the intermittent-catheter system 200 before use of the intermittent catheter 230. The primary chamber 758 and the secondary chamber 760 also respectively provide the primary containment and the secondary containment for any residual urine in or around the intermittent catheter 230, thereby preventing urine leakage from the catheter housing 750 in the storage state of the intermittent-catheter system 200 after use and return of the intermittent catheter 230 to the catheter housing 750 for storage until an entirety of the intermittent-catheter system 200 can be disposed.

Again, the catheter housing 750 can be configured to collapse from the maximally extended state in the storage state of the intermittent-catheter system 200 to the maximally collapsed state in the opened state of the intermittent-catheter system 200 when the closed end portion of the inner sleeve 752 is held against a surface and the outer sleeve 754 is slid over the inner sleeve 752 toward the surface. As to the maximally extended state of the catheter housing 750, an inner perimeter of an outer-sleeve cavity 769 of the outer sleeve 754 steps down in magnitude with an outer-sleeve step 770 toward an opposite end portion of the outer sleeve 754 to that including the end cap 756 (i.e., the distal end portion of the outer sleeve 754 according to the nomenclature used for the intermittent catheter 230). Further, the inner sleeve 752 includes an external ridge 772 configured to abut the outer-sleeve step 770 in the opposite end portion (i.e., the distal end portion) of the outer sleeve 754, thereby defining the maximally extended state of the catheter housing 750. As to the maximally collapsed state of the catheter housing 750, an outer perimeter of the inner sleeve 752 steps up in magnitude with an inner-sleeve step 774 toward the closed end portion of the inner sleeve 752 (i.e., the distal end portion of the inner sleeve 752 according to the nomenclature used for the intermittent catheter 230). A taper of the inner perimeter of the outer-sleeve cavity 769 of the outer sleeve 754 distal of the outer-sleeve step 770 is configured to restrict movement of the inner sleeve 752 up through the end portion of the outer sleeve 754 including the end cap 756 (i.e., the proximal end portion of the outer sleeve 754 according to the nomenclature used for the intermittent catheter 230), thereby defining the maximally collapsed state of the catheter housing 750. Advantageously, the inner-sleeve step 774 in the outer perimeter of the inner sleeve 752 also centers the inner sleeve 752 in the outer sleeve 754 when transitioning between the maximally extended state of the catheter housing 750 in the storage state of the intermittent-catheter system 200 and the maximally collapsed state of the catheter housing 750 in the opened state of the intermittent-catheter system 200.

### Methods of Female Intermittent-Catheter Systems

As set forth below, methods can include methods of using the intermittent-catheter systems and assemblies disclosed herein; however, it should be understood that those methods can be further discerned from description of the intermittent-catheter systems and assemblies and how they are configured to function. Likewise, additional methods can be discerned from description of the intermittent-catheter systems and assemblies and how they are configured to function.

For example, a method of the intermittent-catheter system 200 can include obtaining the intermittent-catheter system 200 in the storage state thereof.

As set forth above, the intermittent-catheter system 200 can include the rigid catheter housing 750 and the intermittent catheter 230.

Briefly, as to the catheter housing 750, the catheter housing 750 can include the inner sleeve 752, the outer sleeve 754 slidably mounted over the inner sleeve 752, and the end cap 756. The end cap 756 can be configured to sit within the end portion of the outer sleeve 754 opposite the closed end portion of the inner sleeve 752 and simultaneously contribute to both the primary chamber 758 for the primary containment and the secondary chamber 760 for the secondary containment. Indeed, the end cap 756 overlaps the open-end portion of the inner sleeve 752, thereby providing the primary chamber 758 between the end-cap cavity 762 of the end cap 756 and the inner-sleeve cavity 764 of the inner sleeve 752. And the end-cap gasket 766 around the end cap 756 and the inner-sleeve gasket 768 around the inner sleeve 752 define longitudinal limits of the secondary chamber 760 between the interior surface of the outer sleeve 754 and the exterior surfaces of the end cap 756 and the inner sleeve 752. Again, the primary chamber 758 and the secondary chamber 760 respectively provide the primary containment and the secondary containment for any lubricant around the catheter tube 234 of the intermittent catheter 230, thereby preventing lubricant leakage from the catheter housing 750 in the storage state of the intermittent-catheter system 200 before use of the intermittent catheter 230.

As to the intermittent catheter 230, the intermittent catheter 230 can include the catheter tube 234 and the funnel 236 over the proximal end portion of the catheter tube 234. The catheter housing 750 can be at the maximally extended state thereof with the intermittent catheter 230 sealed within the primary chamber 758 in the storage state of the intermittent-catheter system 200.

The method can also include transitioning the intermittent-catheter system 200 into the opened state thereof. Transitioning the intermittent-catheter system 200 into the opened state includes grasping the outer sleeve 754 of the catheter housing 750; holding the closed end portion of the inner sleeve 752 of the catheter housing 750 against a surface by way of the outer sleeve 754 of the catheter housing 750; and sliding the outer sleeve 754 of the catheter housing 750 over the inner sleeve 752 of the catheter housing 750 toward the surface to expose the funnel 236 of the intermittent catheter 230. The funnel 236 of the intermittent catheter 230 pops the end cap 756 out of the outer sleeve 754 of the catheter housing 750 when sliding the outer sleeve 754 over the inner sleeve 752 of the catheter housing 750.

The method can also include removing the intermittent catheter 230 from the catheter housing 750 by the funnel 236 of the intermittent catheter 230.

The method can also include inserting the distal end portion of the catheter tube 234 into a urethra; voiding urine from a bladder through the intermittent catheter 230; replacing the intermittent catheter 230 in the inner sleeve 752 of the catheter housing 750; and replacing the end cap 756 within the end portion of the outer sleeve 754, thereby reforming the primary chamber 758 for primary containment of any residual urine in or around the intermittent catheter 230 and the secondary chamber 760 for secondary containment of any leaked urine from the primary chamber 758. Reforming the primary chamber 758 and the secondary chamber 760 thusly prevents urine leakage from the catheter housing 750 in the storage state of the intermittent-catheter system 200 after use of the intermittent catheter 230.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An intermittent-catheter system, comprising:
a rigid catheter housing including:
an inner sleeve;
an outer sleeve slidably mounted over the inner sleeve; and
an end cap configured to sit within an end portion of the outer sleeve opposite a closed end portion of the inner sleeve, the end cap simultaneously contributing to both a primary chamber for primary containment and a secondary chamber for secondary containment; and
an intermittent catheter including a catheter tube and a funnel over a proximal end portion of the catheter tube, the intermittent-catheter system having:
a storage state including the catheter housing at a maximally extended state thereof, the intermittent catheter sealed within the primary chamber; and
an opened state including the outer sleeve of the catheter housing is slid over the inner sleeve of the catheter housing such that the catheter housing is at a maximally collapsed state thereof, the end cap popped out of the outer sleeve and the funnel of the intermittent catheter exposed for removal of the intermittent catheter from the catheter housing.

2. The intermittent-catheter system of claim 1, wherein the end cap overlaps an open-end portion of the inner sleeve, thereby providing the primary chamber between an end-cap cavity of the end cap and an inner-sleeve cavity of the inner sleeve.

3. The intermittent-catheter system of claim 1, wherein an end-cap gasket around the end cap and an inner-sleeve gasket around the inner sleeve define longitudinal limits of the secondary chamber between an interior surface of the outer sleeve and exterior surfaces of the end cap and the inner sleeve.

4. The intermittent-catheter system of claim 1, wherein the primary chamber and the secondary chamber respectively provide the primary containment and the secondary containment for any residual urine in or around the intermittent catheter, thereby preventing urine leakage from the catheter housing in the storage state of the intermittent-catheter system after use of the intermittent catheter.

5. The intermittent-catheter system of claim 3, wherein the end-cap gasket and the inner-sleeve gasket provide sufficient friction for maintaining the storage state of the catheter housing, thereby averting inadvertent collapse into the opened state of catheter housing.

6. The intermittent-catheter system of claim 3, wherein an inner perimeter of an outer-sleeve cavity of the outer sleeve steps down in magnitude with an outer-sleeve step toward an opposite end portion of the outer sleeve to that including the end cap, the inner sleeve including an external ridge configured to abut the outer-sleeve step in the opposite end portion of the outer sleeve, thereby defining the maximally extended state of the catheter housing.

7. The intermittent-catheter system of claim 6, wherein an outer perimeter of the inner sleeve steps up in magnitude with an inner-sleeve step toward the closed end portion of the inner sleeve, a taper of the inner perimeter of the outer-sleeve cavity of the outer sleeve configured to restrict movement of the inner sleeve through the end portion of the outer sleeve including the end cap, thereby defining the maximally collapsed state of the catheter housing.

8. The intermittent-catheter system of claim 7, wherein the inner-sleeve step in the outer perimeter of the inner sleeve centers the inner sleeve in the outer sleeve when transitioning between the storage state and the opened state of the catheter housing.

9. The intermittent-catheter system of claim 1, further comprising a lubricious coating on or a lubricant around the catheter tube of the intermittent catheter sealed within the primary chamber in the storage state of the intermittent-catheter system.

10. The intermittent-catheter system of claim 9, wherein the primary chamber and the secondary chamber respectively provide the primary containment and the secondary containment for the lubricant around the catheter tube of the intermittent catheter, thereby preventing lubricant leakage from the catheter housing in the storage state of the intermittent-catheter system before use of the intermittent catheter.

11. The intermittent-catheter system of claim 1, wherein the catheter housing is configured to collapse when the closed end portion of the inner sleeve is held against a surface and the outer sleeve is slid over the inner sleeve toward the surface, thereby exposing the funnel of the intermittent catheter for removal of the intermittent catheter from the catheter housing.

12. The intermittent-catheter system of claim 1, wherein the funnel includes a plurality of ridges integrated into an outer surface of the funnel, the ridges configured for gripping the funnel as a handle while removing the intermittent catheter from the catheter housing or voiding urine.

13. The intermittent-catheter system of claim 1, wherein the catheter tube includes a plurality of eyelets proximate a catheter tip, the eyelets in fluid communication with the funnel opening.

14. The intermittent-catheter system of claim 1, wherein the intermittent catheter is configured for use by female humans.

15. A method of an intermittent-catheter system, comprising:
obtaining the intermittent-catheter system in a storage state thereof, the intermittent-catheter system including:
a rigid catheter housing including:
an inner sleeve;
an outer sleeve slidably mounted over the inner sleeve; and
an end cap configured to sit within an end portion of the outer sleeve opposite a closed end portion of the inner sleeve, the end cap simultaneously contributing to both a primary chamber for primary containment and a secondary chamber for secondary containment; and
an intermittent catheter including a catheter tube and a funnel over a proximal end portion of the catheter tube, the catheter housing at a maximally extended state thereof with the intermittent catheter sealed within the primary chamber in the storage state of the intermittent-catheter system;
transitioning the intermittent-catheter system into an opened state thereof, the transitioning of the intermittent-catheter system into the opened state including:
grasping an outer sleeve of the catheter housing;
holding a closed end portion of an inner sleeve of the catheter housing against a surface by way of the outer sleeve of the catheter housing; and
sliding the outer sleeve of the catheter housing over the inner sleeve of the catheter housing toward the surface to expose a funnel of the intermittent catheter; and
removing the intermittent catheter from the catheter housing by the funnel of the intermittent catheter.

16. The method of claim 15, wherein the funnel of the intermittent catheter pops the end cap out of the outer sleeve of the catheter housing when sliding the outer sleeve over the inner sleeve of the catheter housing.

17. The method of claim 15, wherein the end cap overlaps an open-end portion of the inner sleeve, thereby providing the primary chamber between an end-cap cavity of the end cap and an inner-sleeve cavity of the inner sleeve.

18. The method of claim 15, wherein an end-cap gasket around the end cap and an inner-sleeve gasket around the inner sleeve define longitudinal limits of the secondary chamber between an interior surface of the outer sleeve and exterior surfaces of the end cap and the inner sleeve.

19. The method of claim 15, wherein the primary chamber and the secondary chamber respectively provide the primary containment and the secondary containment for any lubricant around the catheter tube of the intermittent catheter, thereby preventing lubricant leakage from the catheter housing in the storage state of the intermittent-catheter system before use of the intermittent catheter.

20. The method of claim 15, further comprising:
inserting a distal end portion of the catheter tube into a urethra;
voiding urine from a bladder through the intermittent catheter;
replacing the intermittent catheter in the inner sleeve of the catheter housing; and
replacing the end cap within the end portion of the outer sleeve, thereby reforming the primary chamber for primary containment of any residual urine in or around the intermittent catheter and the secondary chamber for secondary containment of any leaked urine from the primary chamber, which prevents urine leakage from the catheter housing in the storage state of the intermittent-catheter system after use of the intermittent catheter.
